(19) United States
(12) Patent Application Publication
(10) Pub. No.: US 2022/0339227 A1
(43) Pub. Date: Oct. 27, 2022

(54) COMPOSITIONS OF CANNABINOIDS FOR DELIVERY BY INHALATION

(71) Applicants: BOARD OF REGENTS, THE UNIVERSITY OF TEXAS SYSTEM, Austin, TX (US); TFF PHARMACEUTICALS, INC., Austin, TX (US)

(72) Inventors: Robert O. WILLIAMS, III, Austin, TX (US); Chaeho MOON, Austin, TX (US); John J. KOLENG, Austin, TX (US)

(73) Assignees: BOARD OF REGENTS, THE UNIVERSITY OF TEXAS SYSTEM, Austin, TX (US); TFF PHARMACEUTICALS, INC., Austin, TX (US)

(21) Appl. No.: 17/642,295

(22) PCT Filed: Sep. 18, 2020
(86) PCT No.: PCT/US2020/051388

§ 371 Date: Mar. 11, 2022

Related U.S. Application Data
(60) Provisional application No. 62/902,095, filed on Sep. 18, 2019.

(51) Int. Cl. A61K 36/185; A61K 9/00; A61K 47/26; A61K 47/18; A61K 47/28; A61K 31/05

(52) U.S. Cl. CPC A61K 36/185; A61K 9/0075; A61K 47/26; A61K 47/183; A61K 47/28; A61K 31/05

(57) ABSTRACT

The disclosure relates to dry powder compositions of (a) a plant-based cannabidiol (pCBD) composition, or (b) a plant-based tetrahydrocannabinol composition (pTHC); or (c) a synthetic cannabidiol composition (sCBD), or (d) a synthetic tetrahydrocannabinol composition (sTHC), or (e) a plant-based cannabidiol (pCBD) composition in combination with a plant-based tetrahydrocannabinol composition (pTHC), or (f) a synthetic cannabidiol composition (sCBD) in combination with a synthetic tetrahydrocannabinol composition (sTHC) and their delivery to the airway of a subject using a dry powder inhaler.

### PRIORITY CLAIM

The present application claims benefit of priority to U.S. Provisional Application Ser. No. 62/902,095, filed Sep. 18, 2019, the entire contents of which are hereby incorporated by referenced.

### BACKGROUND

### I. Technical Field

The present disclosure relates to thin film preparations of cannabinoids (e.g., cannabidiol (CBD)) in the preparation of dry powders used in administration by inhalation to and/or through the lungs. The preparations are useful in the treatment of various diseases and disorders.

### II. Related Art

Bioavailability of cannabinoids, such as CBD, from oral delivery is low and variable with estimates of about 6% from the World Health Organization. Therefore, higher oral doses are needed for delivery, but this in turn can cause increased chances of adverse effects. For example, when CBD is delivered by intravenous administration, the Cₘₐₓ could be too high, thereby leading to adverse effects. In addition, CBD levels in plasma are not maintained and rapidly decline close to baseline in less than 4 hours when administered intravenously in animals Thus, methods that achieve higher bioavailability for CBD, both in terms of percent of dosage and longevity, would be highly advantageous.

Drug delivery to lung tissue has been achieved using a variety of devices for inhalation, including nebulizers and inhalers, such as metered dose inhalers and dry powder inhalers to treat local and systemic diseases or disorders. Dry powder inhalers used to deliver medicaments to the lungs contain a dose system of a powder formulation usually either in bulk supply or quantified into individual doses stored in unit dose compartments, like hard capsules (e.g., HPMC capsules) or blister packs. Bulk containers are equipped with a measuring system operated by the patient in order to isolate a single dose from the powder immediately before inhalation.

### SUMMARY

The present disclosure concerns thin film freezing (TFF) preparations comprising one or more cannabinoids, one or more excipients, and one or more inactive processing agents. The preparation may be dry powder composition, such as a pharmaceutical inhalation composition. The preparation or the one or more cannabinoids in the preparation may be amorphous. The preparation or the one or more cannabinoids in the preparation may be crystalline, amorphous, or a combination of amorphous and crystalline. The preparation may further comprise a lung surfactant.

The one or more excipients may comprise a sugar or sugar derivative, such as mannitol, trehalose, lactose, sucrose, maltose, a starch, cellulose, xylitol, sorbitol, erythritol, threitol, arabitol, ribitol, galactitol, fucitol, iditol, inositol, volemitol, isomalt, maltitol, lactitol, maltotritol, maltotetraitol, polyglycitol, and/or maltodextrin. The one or more inactive processing agents may be an amino acid or an amino acid derivative such as leucine, arginine, glycine, isoleucine, lysine, valine, methionine, phenylalanine, aspartame, acesulfame K; or other types that are not amino acid or amino acid derivatives such as zinc stearate, magnesium stearate, calcium stearate, povidone K25, and/or sodium stearate. The lung surfactant may comprise one or more of lecithin, oleic acid, lauric acid, palmitic acid, stearic acid, erucic acid, behenic acid, dipalmitoyl phosphatidylcholine (DPPC), dipalmitoyl phosphatidylethanolamine (DPPE), dipalmitoyl phosphatidylinositol (DPPI), phosphatidylcholines, phosphatidylethanolamines, phosphatidylglycerols, sodium lauryl sulphate, magnesium lauryl sulphate, and/or cholesterol. The preparation may also comprise a terpene, terpenes, terpenoids or flavoring agents.

The ratio of cannabinoids:excipient:inactive processing agent may be 10-90:10-90:0-90, 25-35:65-75:0:10 or 25:70:5. The mass median aerodynamic diameter (MMAD) of the particles may be from about 0.5 to about 8 microns, more preferably from about 1 to about 5 microns. The one or more cannabinoids is/are plant-based cannabinoid or synthetic cannabinoids or mixtures thereof. The preparation may further comprise tetrahydro-cannabinol. The one or more cannabinoids may comprise cannabidiol (CBD), for example, where the preparation comprises CBD:mannitol:leucine at a ratio of 25:70:5.

In another embodiment, there is provided a method comprising administering a TFF preparation as defined herein via inhalation. The subject may be a pulmonary disease, such as COPD or asthma. The subject may be a neurological disease or disorder, such as Alzheimer's disease, epilepsy, an autism spectrum disorder, PTSD, Parkinson's disease, Huntington's disease, schizophrenia, stroke, major depression or traumatic brain injury. The subject may be ocular disease, such as macular degeneration, glaucoma or retinitis pigmentosa (RP). The subject may have cancer, Rett syndrome (RTT), Lennox-Gastaut Syndrome (LGS), Tuberous Sclerosis Complex (TSC), Dravet syndrome, nausea, anxiety, pain, dystonia, diabetes, Rheumatoid arthritis, Crohn's disease, graft-versus-host disease (GVHD) or HIV infection.

Administering may employ a dry powder inhaler device comprising said TFF preparation. The TFF preparation may be administered 1, 2, 3, 4, 5, 6, 7, 8, 9, 10, 11, 12, 13 or 14 times a week or 1, 2, 3, 4, 5 or 6 times per day, or is administered on a chronic basis. The subject may be treated with at least another therapy, such as where the other therapy is (a) given through a pulmonary route, (b) given through a non-pulmonary route, (c) given before, at the same time or after the TFF preparation, (d) co-formulated with the TFF preparation, or (e) not co-formulated with the TFF preparation. The other therapy may be a second inhalation therapy, and the second inhalation therapy is administered using the same dry powder inhaler as the TFF preparation but in a distinct compartment from said TFF preparation. Aa single dose of said TFF preparation may be 0.1 mg to 100 mg. A total dose of said TFF preparation may be 0.1 to 10 g.

In yet another embodiment, there is provided a method of preparing a thin film freezing (TFF) preparation comprising one or more cannabinoids, one or more excipients, one or more inactive processing agents comprising (a) mixing one or more cannabinoids, one or more excipients, and one or more inactive processing agents in one or more solvents to produce a solution; (b) applying said solution to a rotating drum cooled to −60° C. or lower to produce a frozen solution; (c) collecting the frozen solution; (d) storing said frozen solution at −80° C. to remove residue liquid nitrogen; and (3) removing solvent by sublimation. The preparation may further comprise a lung surfactant and/or a terpene/terpenes and/or a flavoring agent. The one or more cannabinoids may comprise cannabidiol (CBD), for example, where the preparation comprises CBD:mannitol:leucine at a ratio of 25:70:5.

A dry powder of the embodiments is produced by thin film freezing (TFF), see, e.g., US20100221343, which is incorporated herein by reference.

Also disclosed is a dry powder inhaler comprising a TFF preparation as defined here. The dry powder inhaler may be a breath-powered inhaler, is compact, may be reusable or disposable, may be various shapes and sizes, and comprises a system of airflow conduit pathways for the effective and rapid delivery of powder medicament to the lungs and/or the systemic circulation.

In one embodiment, the dry powder inhaler comprises a unit dose cartridge, and a dry powder formulation that is to be aerosolized and delivered to lung tissue for a local tissue effect, or for absorption into the blood stream in the lungs and be delivered by the systemic circulation to target tissue or organs of a subject. In an embodiment, the dry powder can comprise, a carrier molecule, including pharmaceutically acceptable carriers and excipients, for example, phospholipids, polymers such as polyethylene glycol, co-glycolides, a saccharide, a polysaccharide, and an active ingredient.

The dry powder may comprise an inhalable dry powder, including a pharmaceutical formulation comprising a cannabis agent (e.g., one or more cannabinoids) for pulmonary delivery. In some embodiments, delivery is to the deep lung (that is, to the alveolar region) and in some of these embodiments, the active agent or active ingredient is absorbed into the pulmonary circulation for systemic targeted or general use.

Cartridges for use with the dry powder inhaler can be manufactured to contain the dry powder medicament for inhalation. In one embodiment, the cartridge is structurally configured to be adaptable to a particular dry powder inhaler and can be made of any size and shape, depending on the size and shape of the inhaler to be used with, for example, if the inhaler has a mechanism which allows for translational movement or for rotational movement.

In some embodiments, the dry powder formulation is dispensed with consistency from the inhaler in less than about three (3) seconds, or generally less than one (1) second. In some embodiments, the inhaler air conduits are designed to yield high resistance to air flow values of, for example, approximately 0.065 to about 0.200 (kPa)/liter per minute. Therefore, in the inhalation system, peak inhalation pressure drops of between 2 and 20 kPa produce resultant peak flow rates of about between 7 and 70 liters per minute. These flow rates result in greater than 75% of the cartridge contents dispensed in fill masses between 1 and 50 mg. In some embodiments, these performance characteristics are achieved by end users within a single inhalation maneuver to produce cartridge dispense percentage of greater than 90%. In certain embodiments, the inhaler and cartridge system are configured to provide a single dose by discharging powder from the inhaler as a continuous flow, or as one or more pulses of powder delivered to a patient.

One of ordinary skill in the art will appreciate that starting materials, biological materials, reagents, synthetic methods, purification methods, analytical methods, assay methods, and biological methods other than those specifically exemplified can be employed in the practice of the disclosure without resort to undue experimentation. All art-known functional equivalents, of any such materials and methods are intended to be included in this disclosure.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by particular embodiments and optional features, modification and variation of the concepts herein disclosed may be resorted to by those skilled in the art, and that such modifications and variations are considered to be within the scope of this disclosure as defined by the appended claims.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Scanning Electron Microscope images of TFF-Cannabinoid. Scanning electron microscopy (SEM) was used to assess the dry powder morphology of the TFF-CDB prepared in Example 1. The SEM micrographs are for formulations 6, 7, 10, 11, 12 and 13

FIG. 2. X-ray powder diffraction data of TFF-Cannabinoids. X-ray Powder Diffraction (XRPD) shown was used to assess the morphology of the TFF-CBD dry powders. Crystalline and amorphous TFF-CBD powders for inhalation were prepared as described in Example 1. The order of the formulations on the right hand edge corresponds to the same order as the diffraction tracings.

FIG. 3. Cannabidiol concentrations in rat plasma.

FIG. 4. Cannabidiol concentrations in rat plasma.

FIG. 5. Scanning electron microscopy of formulation 26 dry powder. Exemplary formulation 26 dry powder suitable for inhalation prepared as described in Example 1 was further tested.

FIG. 6. X-ray powder diffraction for formulation 26 dry powder. Exemplary formulation 26 dry powder suitable for inhalation prepared as described in Example 1 was further tested.

FIG. 7. Aerodynamic particle size distribution for formulation 26 dry powder contained in an HPMC capsule and using the Plastiape dry powder inhaler device. Exemplary formulation 26 dry powder suitable for inhalation prepared as described in Example 1 was further tested. Plastiape high resistance RS01 device, 69 L/min, 4 kPa pressure drop (n=3). The following aerodynamic properties were determined: MMAD: 3.59±0.18; GSD: 2.59±0.24; FPF (% of recovered): 47.29±4.41; FPF (% of delivered): 51.14±6.65; delivered dose (%): 92.77±3.31.

FIG. 8. Comparison of PK Data to Hložek et al. (2017). 7.98 mg/kg of vaporized Cannabidiol was administered. The Cannabidiol level in rat serum declined below 20 ng/mL in 4 hours and continue to decrease between 4 and 24 hours in Hložek et al. (2017).

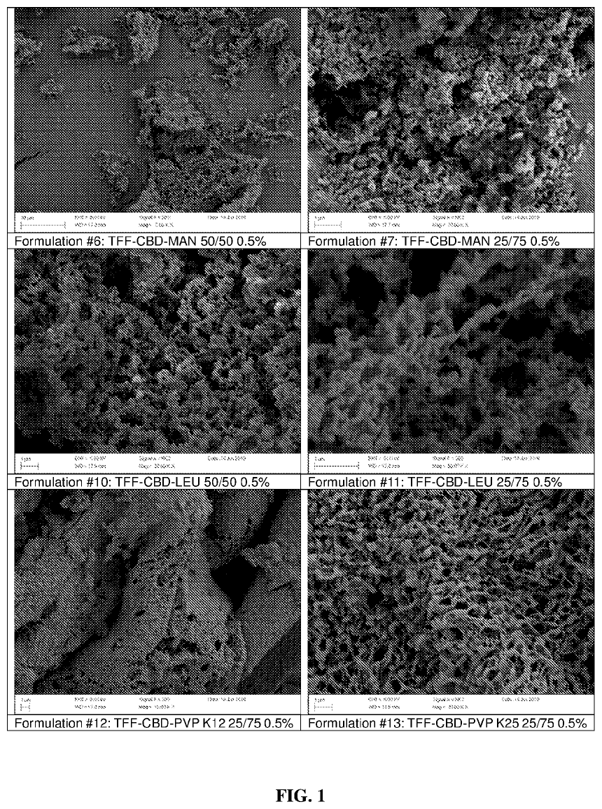

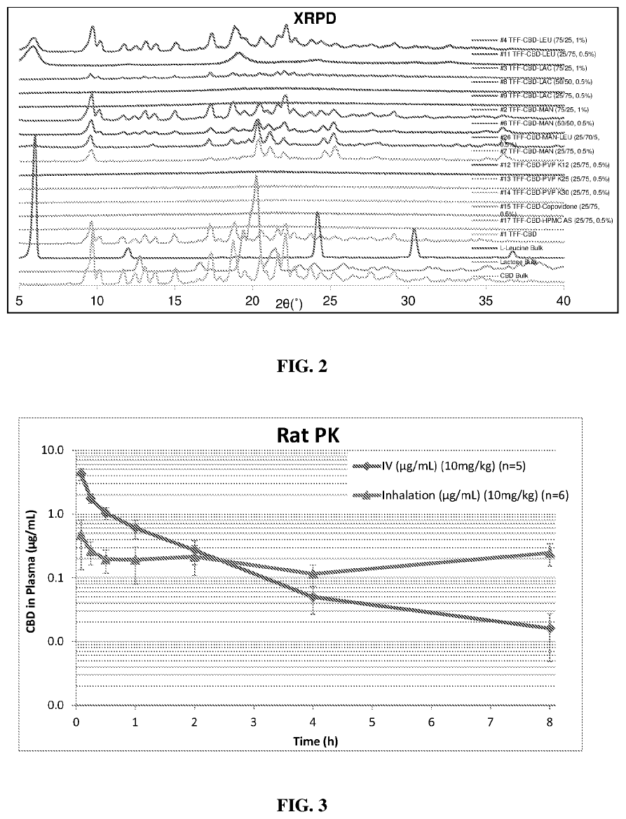

### DETAILED DESCRIPTION

In embodiments disclosed herein, cannabinoid compositions prepared by TFF techniques are presented as are their utility for delivery to subjects with improved efficiency and duration. In particular, the materials include particular excipients and ratios thereof. Further details regarding the disclosure are set out below.

### I. Definitions

As used herein the term “a unit dose inhaler” refers to an inhaler that is adapted to receive a single cartridge or container comprising a dry powder formulation and delivers a single dose of a dry powder formulation by inhalation from a single container to a user. It should be understood that in some instances multiple unit doses will be required to provide a user with a specified dosage.

As used herein a “cartridge” is an enclosure configured to hold or contain a dry powder formulation, a powder containing enclosure, which has a cup or container and a lid. The cartridge is made of rigid materials, and the cup or container is moveable relative to the lid in a translational motion or vice versa.

As used herein a “powder mass” is referred to an agglomeration of powder particles or agglomerate having irregular geometries such as width, diameter, and length.

As used herein a “unit dose” refers to a pre-metered dry powder formulation for inhalation. Alternatively, a unit dose can be a single container having multiple doses of formulation that can be delivered by inhalation as metered single amounts. A unit dose cartridge/container contains a single dose. Alternatively, it can comprise multiple individually accessible compartments, each containing a unit dose.

As used herein, the term “about” is used to indicate that a value includes the standard deviation of error for the device or method being employed to determine the value; and indicates a difference of less than +/−10% of the stated value.

As used herein, the term “microparticle” refers to a particle with a diameter of about 0.5 to about 1000 μm, irrespective of the precise exterior or interior structure. Microparticles having a diameter of between about 0.5 and about 10 microns can reach the lungs, successfully passing most of the natural barriers. A diameter of less than about 10 microns is required to navigate the turn of the throat and a diameter of about 0.5 μm or greater is required to avoid being exhaled. To reach the deep lung (or alveolar region) where most efficient absorption is believed to occur, it is preferred to maximize the proportion of particles contained in the “respirable fraction” (RF), generally accepted to be those particles with an aerodynamic diameter of about 0.5 to about 6 μm, though some references use somewhat different ranges, as measured using standard techniques, for example, with an Anderson Cascade Impactor. Other impactors can be used to measure aerodynamic particle size such as the NEXT GENERATION IMPACTOR® (NGI®, MSP Corporation), for which the respirable fraction is defined by similar aerodynamic size, for example <6.4 μm. In some embodiments, a laser diffraction apparatus is used to determine particle size, for example, the laser diffraction apparatus disclosed in U.S. Pat. No. 8,508,732, which disclosure is incorporated herein in its entirety for its relevant teachings related to laser diffraction, wherein the volumetric median geometric diameter (VMGD) of the particles is measured to assess performance of the inhalation system. For example, in various embodiments cartridge emptying of >80%, 85%, or 90% and a VMGD of the emitted particles of <12.5 μm, <7.0 μm, or <4.8 μm can indicate progressively better aerodynamic performance.

Percent emitted dose represents the percentage (%) of powder in a dose that is emitted from an inhaler upon discharge of the powder content filled for use as the dose, and that is suitable for respiration, i.e., the percent of particles from the filled dose that are emitted with sizes suitable for pulmonary delivery, which is a measure of microparticle aerodynamic performance A % emitted dose value of 40% or greater than 40% reflects acceptable aerodynamic performance characteristics. In certain embodiments disclosed herein, the % emitted dose can be greater than 50%. In an exemplary embodiment, a % emitted dose can be up to about 80%, wherein about 80% of the fill is emitted with particle sizes <5.8 μm as measured using standard techniques.

As used herein, the term “dry powder” refers to a fine particulate composition that is not suspended or dissolved in a propellant, or other liquid. It is not meant to necessarily imply a complete absence of all water or solvent molecules.

As used herein, “amorphous powder” refers to dry powders lacking a definite repeating form, shape, or structure, including all non-crystalline powders.

### II. Thin Film Freezing

The materials of the present disclosure may be prepared using convention methods such as spray freeze drying or thin film freezing as described herein and in U.S. Patent Application No. 2010/0221343 and Watts, et al. (2013), both of which are incorporated herein by reference. After freezing, these particles may be further subjected to drying to obtain a dry powder suitable for aerosol administration. The powder may be dried through lyophilization and other methods known to those of skill in the art.

In some embodiments, the methods comprise dissolving the materials in a solvent. Some solvents which may be used in the methods described herein include water, an organic solvent, or a mixture thereof. The organic solvents that may be used herein include polar organic solvents such an alcohol, a heterocyclic compound, an alkylnitrile, or a mixture thereof. Some non-limiting examples of polar organic solvents include methanol, ethanol, isopropanol, tert-butanol (tertiary butanol), dimethylsulfoxide, dimethylformamide, 1,4-dioxane, or acetonitrile. In some aspects, mixtures of these solvents are contemplated. Such mixtures may comprise one or more organic solvents with water. One non-limiting example of these mixtures includes the solvent mixture of tert-butanol, 1,4-dioxane, acetonitrile, and water. The solvent mixture may comprise a mixture of tertiary butanol, 1,4-dioxane, acetonitrile, and purified water in a ratio of 2:1:3:3 (v/v).

In some aspects, the present disclosure comprises a combination of two or more active ingredients such as THC and cannabinoids. These combinations may further comprise one or more excipients. Some non-limiting examples of some excipients which may be used herein include a a sugar or sugar derivative, such as mannitol, trehalose, lactose, sucrose, maltose, a starch, cellulose, xylitol, sorbitol, erythritol, threitol, arabitol, ribitol, galactitol, fucitol, iditol, inositol, volemitol, isomalt, maltitol, lactitol, maltotritol, maltotetraitol, polyglycitol, and/or maltodextrin. The composition may comprise one or more flavoring agent. These compositions may be dissolved in a solvent as described herein.

Another useful additive for the described composition is a terpene. Terpenes are a large and diverse class of organic compounds, produced by a variety of plants, particularly conifers, and by some insects. They often have a strong odor and may protect the plants that produce them by deterring herbivores and by attracting predators and parasites of herbivores. Although sometimes used interchangeably with “terpenes”, terpenoids (or isoprenoids) are modified terpenes as they contain additional functional groups, usually oxygen-containing. Terpenes are hydrocarbons.

Terpenes and terpenoids are the primary constituents of the essential oils of many types of plants and flowers. Essential oils are used widely as fragrances in perfumery and traditional medicine, such as aromatherapy. Synthetic variations and derivatives of natural terpenes and terpenoids also greatly expand the variety of aromas used in perfumery and flavors used in food additives. Vitamin A is a terpenoid. Terpenes may be classified by the number of isoprene units in the molecule; a prefix in the name indicates the number of terpene units needed to assemble the molecule.
Hemiterpenes consist of a single isoprene unit. Isoprene itself is considered the only hemiterpene, but oxygen-containing derivatives such as prenol and isovaleric acid are hemiterpenoids.
Monoterpenes consist of two isoprene units and have the molecular formula C₁₀H₁₆. Examples of monoterpenes and monoterpenoids include geraniol, terpineol (present in lilacs), limonene (present in citrus fruits), myrcene (present in hops), linalool (present in lavender) or pinene (present in pine trees). Iridoids derive from monoterpenes.
Sesquiterpenes consist of three isoprene units and have the molecular formula C₁₅H₂₄. Examples of sesquiterpenes and sesquiterpenoids include humulene, farnesenes, farnesol.
Diterpenes are composed of four isoprene units and have the molecular formula C₂₀H₃₂. They derive from geranylgeranyl pyrophosphate. Examples of diterpenes and diterpenoids are cafestol, kahweol, cembrene and taxadiene (precursor of taxol). Diterpenes also form the basis for biologically important compounds such as retinol, retinal, and phytol.
Sesterterpenes, terpenes having 25 carbons and five isoprene units, are rare relative to the other sizes. An example of a sesterterpenoid is geranylfarnesol.
Triterpenes consist of six isoprene units and have the molecular formula C₃₀H₄₈. The linear triterpene squalene, the major constituent of shark liver oil, is derived from the reductive coupling of two molecules of farnesyl pyrophosphate. Squalene is then processed biosynthetically to generate either lanosterol or cycloartenol, the structural precursors to all the steroids.
Sesquarterpenes are composed of seven isoprene units and have the molecular formula C₃₅H₅₆. Sesquarterpenes are typically microbial in their origin. Examples of sesquarterpenoids are ferrugicadiol and tetraprenylcurcumene.
Tetraterpenes contain eight isoprene units and have the molecular formula C₄₀H₆₄. Biologically important tetraterpenoids include the acyclic lycopene, the monocyclic gamma-carotene, and the bicyclic alpha- and beta-carotenes.
Polyterpenes consist of long chains of many isoprene units. Natural rubber consists of polyisoprene in which the double bonds are cis. Some plants produce a polyisoprene with trans double bonds, known as gutta-percha.
Norisoprenoids, such as the C₁₃-norisoprenoids 3-oxo-α-ionol present in Muscat of Alexandria leaves and 7,8-dihydroionone derivatives, such as megastigmane-3,9-diol and 3-oxo-7,8-dihydro-α-ionol found in Shiraz leaves (both grapes in the species Vitis vinifera) or wine (responsible for some of the spice notes in Chardonnay), can be produced by fungal peroxidases or glycosidases.

Indeed, the majority of cannabidiol studies in animals employ a single synthetic molecule, CBD. In contrast, whole plant extracts typically include CBD, THC, and hundreds of other compounds, many of which interact with each other to create what is referred to as an “entourage effect” that magnifies the therapeutic activity of individual components. It is important to consider this effect (or lack thereof) when attempting to interpret data from animal studies. For example, the pharmacological importance of terpenes, or terpenoids, is highlighted by their role in aromatherapy, a popular holistic healing modality, and from the fact that marijuana's fragrance and psychoactive flavor are determined by the particular terpenes present in a given plant strain. Around 200 terpenes have been found in cannabis, but only a few appear in relevant amounts. Among those are monoterpenes, diterpenes, and sesquiterpenes. Particular terpenes of interest in cannabis are limonene, pinene, linalool, caryophyllene, and humulene.

In some aspects, the compositions are prepared using a thin film apparatus. The apparatus may be used to apply the solution to a surface such as a stainless steel and then frozen. This surface may also be rotating such that without wishing to be bound by any theory, it is believed that the rotation prompts the even application of the solution to the surface. The solution may be frozen at a cryogenic temperature such as a temperature below −50° C. Cryogenic temperatures include a temperature form about −50° C. to about −270° C., form about −70° C. to about −120° C., or form about −75° C. to about −100° C. In some embodiments, the cryogenic temperature is about −90° C. In some aspects, the samples are stored frozen. In other aspects, the samples are lyophilized to obtain a dry powder. Lyophilization is known to those of skill in the art and is taught in U.S. Pat. Nos. 5,756,468, 6,440,101, 8,579,855, and PCT Patent Application Publication No. WO 2009/125986, which are incorporated herein by reference. In some aspects, it may be advantageous to store the composition at room temperature. The lyophilized samples may be prepared such that the temperature is gradually increased from the lyophilization temperature of less than −40° C. to a temperature around room temperature such as about 25° C. Also, the increase in temperature may be carried out under a vacuum or in a reduced pressure environment and/or an environment which has a reduced moisture content such as a desiccator.

The materials described herein may inactive processing agents. Such agents include an amino acid or an amino acid derivative such as leucine, arginine, glycine, isoleucine, lysine, valine, methionine, phenylalanine, aspartame, acesulfame K; or other agents that are not amino acid or amino acid derivatives such as zinc stearate, magnesium stearate, calcium stearate, povidone K25, polysorbate 80, and/or sodium stearate.

The materials described herein my include a lung-surfactant, such as one or more of lecithin, oleic acid, lauric acid, palmitic acid, stearic acid, erucic acid, behenic acid, dipalmitoyl phosphatidylcholine (DPPC), dipalmitoyl phosphatidylethanolamine (DPPE), dipalmitoyl phosphatidylinositol (DPPI), phosphatidylcholines, phosphatidylethanolamines, phosphatidylglycerols, sodium lauryl sulphate, magnesium lauryl sulphate, a polysorbate and/or cholesterol.

### III. Inhaler Devices

The dry powder inhalers disclosed herein may be of various shapes and sizes, and can be reusable, easy to use, inexpensive to manufacture and/or produced in high volumes in simple steps using plastics or other acceptable materials. Various embodiments of the dry powder inhalers are provided herein and in general, the inhalation systems comprise inhalers, powder-filled cartridges, and empty cartridges. The present inhalation systems can be designed to be used with any type of dry powder. In one embodiment, the dry powder is a relatively cohesive powder which requires optimal deagglomeration conditions.

Commercially available multi-dose inhalers include FLOVENT® DISKUS, ADVAIR® DISKUS, and PULMICORT® FLEXHALER, to name a few. For example, the AFREZZA® inhaler is a unit dose dry powder inhaler, which delivers a human insulin formulation for the treatment of diabetes in humans. AFREZZA was approved by the U.S. Food and Drug Administration for the treatment of diabetes type 1 and type 2 in June 2014. The AFREZZA inhaler is a breath-actuated, multiple use inhaler which delivers a single dose of insulin contained in a cartridge to the lungs, wherein the insulin is absorbed into the circulation for the effective treatment of hyperglycemia associated with diabetes.

Dry powder inhalers such as those described in U.S. Pat. Nos. 7,305,986, 7,464,706, 8,499,757, 8,636,001, and U.S. Patent Publication No. 20170216538, which disclosures are incorporated herein by reference in their entirety, can generate primary drug particles, or suitable inhalation plumes during an inspiratory maneuver by deagglomerating the powder formulation within a capsule or cartridge comprising a single dose. The amount of fine powder discharged from the inhaler's mouthpiece during inhalation is largely dependent on, for example, the inter-particulate forces in the powder formulation and the efficiency of the inhaler to separate those particles so that they are suitable for inhalation. The benefits of delivering drugs via the pulmonary circulation are numerous and include rapid entry into the arterial circulation, avoidance of drug degradation by liver metabolism, and ease of use without discomfort.

In exemplary embodiments herewith, the present devices can be manufactured by several methods and from various materials. In one embodiment, the inhalers and cartridges are made, for example, by injection molding techniques, thermoforming, blow molding, pressing, 3D printing, and the like using various types of plastic materials, including, polypropylene, cyclicolephin co-polymer, nylon, and other compatible polymers and the like. In certain embodiments, the dry powder inhaler can be assembled using top-down assembly of individual component parts. In some embodiments, the inhalers are generally provided in compact sizes, for example, from about 1 inch to about 5 inches in dimension, and generally, the width and height are less than the length of the device. In certain embodiments the inhaler is provided in various shapes including, relatively rectangular bodies, although other shapes can be used such as cylindrical, oval, tubular, squares, oblongs, and circular forms.

The inhalers effectively fluidize, deagglomerate or aerosolize a dry powder formulation by using at least one relatively rigid flow conduit pathway for allowing an airflow to enter the inhaler. For example, the inhaler is provided with a first air flow pathway for entering and exiting a cartridge containing the dry powder, and a second air pathway which can merge with the first air flow pathway exiting the cartridge. The flow conduits, for example, can have various shapes and sizes depending on the inhaler configuration. In one embodiment, inhalers are high resistance inhalers with resistance value of, for example, approximately 0.065 to about 0.200 (kPa)/liter per minute. Therefore, in the system, peak inhalation pressure drops of between 2 and 20 kPa produce resultant peak flow rates of about between 7 and 70 liters per minute. These flow rates result in greater than 75% of the cartridge contents dispensed in fill masses between 1 and 50 mg. In some embodiments, these performance characteristics are achieved by end users within a single inhalation maneuver to produce cartridge dispense percentage of greater than 90% of the powder contained in a cartridge.

Cartridge embodiments for use with the inhalers are described in U.S. Pat. No. 8,424,518, which disclosure is incorporated by reference in its entirety. In summary, a cartridge for use with the inhaler embodiments disclosed herewith comprises two parts, although other embodiments may be envisioned. The cartridges are configured to contain a dry powder medicament in a storage, tightly sealed or contained position and can be reconfigured within an inhaler from a powder containment position to an inhalation or dosing configuration. In certain embodiments, the cartridge comprises a lid and a cup having one or more apertures, a containment configuration and dosing configuration, an outer surface, an inner surface defining an internal volume; and the containment configuration restricts communication to the internal volume and the dispensing configuration forms an air passage through said internal volume to allow an air flow to enter and exit the internal volume in a predetermined manner. For example, the cartridge container can be configured so that an airflow entering the cartridge air inlet is directed across the air outlets within the internal volume to meter the medicament leaving the cartridge so that rate of discharge of a powder is controlled; and wherein airflow in the cartridge can tumble substantially perpendicular to the air outlet flow direction, mix and fluidize a powder in the internal volume prior to exiting through dispensing apertures. Cartridges for use with the instant inhalers can be provided in individual blisters or grouped in a blister depending in the need of the subject or the hygroscopicity of the formulation with respect to stability of powder and/or the active ingredient.

In embodiments, the dry powder inhaler and cartridge form an inhalation system which can be structurally configured to effectuate a tunable or modular airflow resistance, as it can be effectuated by varying the cross-sectional area or geometries of the air conduits at any section of the airflow pathway of the system. In one embodiment, the dry powder inhaler system geometries of the air conduits can generate an airflow resistance value of from about 0.065 to about 0.200 (kPa)/liter per minute. In other embodiments, a check valve may be employed to prevent air flow through the inhaler until a desired pressure drop, such as 4 kPa has been achieved, at which point the desired resistance reaches a value within the range given herewith.

In yet another embodiment, an inhalation system for delivering a dry powder formulation to a patient is provided. The system comprises an inhaler including a container mounting area configured to receive a container and a mouthpiece having at least two inlet apertures and at least one exit aperture; wherein one inlet aperture of the at least two inlet apertures is in communication with the container area, and one of the at least two inlet apertures is in communication with the at least one exit aperture via a flow path configured to bypass the container area to deliver the dry powder formulation to the patient; wherein the flow conduit configured to bypass the container area delivers 30% to 90% of the total flow going through the inhaler during an inhalation.

In another embodiment, a dry powder inhalation system for delivering a dry powder formulation to a patient is also provided. The system comprises a dry powder inhaler including a mounting and reconfiguring region for a cartridge; said dry powder inhaler and cartridge combined are configured to have at least two airflow pathways which are rigid flow conduits in a dosing configuration and a plurality of structural regions that provide a mechanism for powder deagglomeration of the inhalation system in use; wherein at least one of the plurality of mechanisms for deagglomeration is an agglomerate size exclusion aperture in the container region having a smallest dimension between 0.5 mm and 3 mm.

In embodiments disclosed herein, a dry powder formulation can consist of a crystalline powder, an amorphous powder, or combinations thereof, wherein the powder is dispensed with consistency from the inhaler in less than about 2 seconds. The present inhaler system has a high resistance value of approximately 0.065 to about 0.200 (kPa)/liter per minute. Therefore, in the system comprising a cartridge, peak inhalation pressure drops applied of between 2 and 20 kPa produce resultant peak flow rates of about through the system of between 7 and 70 liters per minute. These flow rates result in greater than 75% of the cartridge contents dispensed in fill masses between 1 and 30 mg, or up to 50 mg of powder. In some embodiments, these performance characteristics are achieved by end users within a single inhalation maneuver to produce cartridge dispense percentage of greater than 90%. In certain embodiments, the inhaler and cartridge system are configured to provide a single dose by discharging powder from the inhaler as a continuous flow, or as one or more pulses of powder delivered to a patient. In an embodiment, an inhalation system for delivering a dry powder formulation to a patient's lung(s) is provided, comprising a dry powder inhaler configured to have flow conduits with a total resistance to flow in a dosing configuration ranging in value from 0.065 to about 0.200 (kPa)/liter per minute. In this and other embodiments, the total resistance to flow of the inhalation system is relatively constant across a pressure differential range of between 0.5 kPa and 7 kPa.

The structural configuration of the inhaler can permit the deagglomeration mechanism to produce an emitted dose greater than 50% and particles of less than 5.8 μm aerodynamic diameter. The inhalers can discharge greater than 85% of a powder medicament contained within a container during an inhalation maneuver. Generally, the inhalers herein depicted herewith can discharge greater than 90% of the cartridge contents or container (e.g., from capsule and device) contents in less than 3 seconds at pressure differentials between 2 and 5 kPa with fill masses ranging up to 30 mg or 50 mg.

While inhalers are primarily described as breath-powered, in some embodiments, the inhaler can be provided with a source for generating the pressure differential required to deagglomerate and deliver a dry powder formulation. For example, an inhaler can be adapted to a gas-powered source, such as compressed gas stored energy source, such as from a nitrogen can, which can be provided at the air inlet ports. A spacer can be provided to capture the plume so that the patient can inhale at a comfortable pace.

In embodiments, the inhaler can be provided as a reusable inhaler for delivering a single unit dose. A reusable inhaler means that it can be used multiple times which can be predetermined depending on the formulation to be delivered and discarded once it has reached its maximal usage. Alternatively, the dry powder inhaler is reusable and is provided with a replaceable cartridge for single use to deliver a single dose using a single inhalation provided by a subject. In this embodiment, multiple cartridges of a specific powder content containing an active ingredient and packaged, for example, in a blister pack can be provided with a single inhaler for multiple uses by a subject. In this and other embodiments, a cartridge can comprise a dry powder formulation for treating a variety of conditions, diseases or disorders.

A system for the delivery of an inhalable dry powder is also provided, comprising: a) a dry powder comprising a medicament, and b) an inhaler comprising a powder containing cartridge, the cartridge comprising a gas inlet and a gas outlet, and a housing in which to mount the cartridge and defining two flow pathways, a first flow pathway allowing gas to enter the gas inlet of the cartridge, a second flow pathway allowing gas to bypass the enclosure gas inlet, and a mouthpiece and upon applying a pressure drop of >2 kPa across the inhaler plume of particles is emitted from the mouthpiece wherein 50% of said emitted particles have a VMAD of <10 μm, wherein flow bypassing the cartridge gas inlet is directed to impinge upon the flow exiting the enclosure substantially perpendicular to the gas outlet flow direction.

An inhalation system for delivering a dry powder formulation to a patient's lung(s) is provided, the system comprising a dry powder inhaler configured to have flow conduits with a total resistance to flow in a dosing configuration ranging in value from 0.065 to about 0.200 (kPa)/liter per minute.

### IV. Powder Formulations and Methods of Making the Same

These present devices and systems are useful in pulmonary delivery of powders with a wide range of characteristics. Embodiments include systems comprising an inhaler, an integral or installable unit dose cartridge comprising the desirable powder doses. Pulmonary delivery of powders can include carriers and excipients which safety and efficacy have been proven in commercially available products. In some cases, the active agent(s) can be formulated without the use of a carrier or excipient. Dry powders can be made by lyophilizing, or spray-drying solution or suspensions of the various desired formulations. Crystalline microparticles and amorphous microparticles with a specific surface area (SSA) of between about 35 and about 67 m²/g exhibit characteristics beneficial to delivery of drugs to the lungs such as improved aerodynamic performance and improved drug adsorption. In some embodiments, high capacity crystalline microparticles or amorphous microparticles have a specific surface area which is less than 35 m²/g and specific surface area of these particles can range from about 19 m²/g to about 30 m²/g or from about 28 m²/g to about 71 m²/g, or from about 19 m²/g to about 57 m²/g depending on the amount of active agent. In some embodiments, microparticles can have specific surface area ranging from about 4 m²/g to about 30 m²/g and have improved aerodynamic properties as measured by aerodynamic properties and flowability of the powders.

In one embodiment, the dry powder medicament may form particles, microparticles and aggregates of microparticles and the like, herein referred to as microparticles, which can be used as carrier systems for the delivery of active agents to a target site in the body. The term “active agent” is referred to herein as the therapeutic agent that can be incorporated into a carrier or formulated without a carrier. The dry powder medicament can be used to deliver biologically active agents having therapeutic, prophylactic or diagnostic activities.

Microparticles for pulmonary delivery having an aerodynamic diameter of between about 0.5 and about 10 μm can reach the lungs and can reach the systemic circulation and deliver an active agent. A diameter of less than about 10 μm is required to navigate the turn of the throat and a diameter of about 0.5 μm or greater is required to avoid being exhaled. Generally, microparticles having diameters greater than 10 μm or greater than 20 μm are useful for local delivery to the respiratory tract and lungs. Unless noted otherwise, diameter means aerodynamic diameter.

Microparticles having a diameter of between about 0.5 and about 10 microns can reach the lungs, successfully passing most of the natural barriers. A diameter of less than about 10 microns is required to navigate the turn of the throat and a diameter of about 0.5 microns or greater is required to avoid being exhaled. Microparticles with a specific surface area (SSA) of between about 4 and about 71 m²/g may exhibit characteristics beneficial to delivery of drugs to the lungs such as improved aerodynamic performance and improved drug adsorption.

In certain embodiments, a composition for pulmonary delivery is provided with an active agent comprises a plurality of substantially uniformly formed, microparticles, wherein the particles have a substantially hollow spherical structure and comprise a shell that do not self-assemble, and the particles have a volumetric mean geometric diameter less than equal to 5 μm; wherein the particles are formed by a method comprising the step of combining an excipient in a solution without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus.

The microparticles can have a substantially hollow spherical structure and comprise a shell which can be porous. In certain embodiments, the microparticles can be substantially hollow spherical and substantially solid particles comprising the drug and/or drug content provided and other factors in the process of making the powders. In one embodiment, the microparticles comprise particles that are relatively porous, having average pore volumes of about 0.43 cm³/g, ranging from about 0.4 cm³/g to about 0.45 cm³/g, and average pore size ranging from about 23 nm to about 30 nm, or from about 23.8 nm to 26.2 nm as determined by techniques known in the art such as BJH adsorption or mercury porosimetry.

In a particular embodiment herein, up to about 92% of the microparticles have a volumetric median geometric diameter of 5.8 μm.

The method can further comprise the steps of adding with mixing a solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying.

Formation of the composition comprises the step wherein the material comprising the active agents is optionally filtered or winterized to separate and remove layers of unwanted materials such as lipids to increase its solubility.

The method can further comprise the steps of adding with mixing a solution, the mixing can optionally be performed with or without homogenization in a high shear mixer, the solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying, or the particles can be formed from the solution during spray-drying.

In some embodiments herewith, the cannabinoid content can be from about 0.01% (w/w) to about 75% (w/w); from about 1% to about 50% (w/w), from about 10% (w/w) to about 25% (w/w), or from about 10% to about 20% (w/w), or from 5% to about 30%, or greater than 25%.

### V. Cannabis Active Agents

The active agents for use in the compositions and methods described herein include a cannabis agent. These can include both plant-based and synthetic materials. The plant-based materials can be obtained from plants or chemically produced to provide the same chemical compound as that found in the plant-based materials.

1. Plant-Based Materials Cannabis is a genus of flowering plants in the family Cannabaceae. The number of species within the genus is disputed. Three species may be recognized: Cannabis sativa, Cannabis indica, and Cannabis ruderalis; C. ruderalis may be included within C. sativa; all three may be treated as subspecies of a single species, C. sativa; or C. sativa may be accepted as a single undivided species. The genus is widely accepted as being indigenous to and originating from Central Asia, with some researchers also including upper South Asia in its origin.

The plant is also known as hemp, although this term is often used to refer only to varieties of Cannabis cultivated for non-drug use. Cannabis has long been used for hemp fibre, hemp seeds and their oils, hemp leaves for use as vegetables and as juice, medicinal purposes, and as a recreational drug. Industrial hemp products are made from cannabis plants selected to produce an abundance of fiber. To satisfy the UN Narcotics Convention, some cannabis strains have been bred to produce minimal levels of tetrahydrocannabinol (THC), the principal psychoactive constituent. Some strains have been selectively bred to produce a maximum of THC (a cannabinoid), the strength of which is enhanced by curing the flowers. Various compounds, including hashish and hash oil, are extracted from the plant.

Globally in 2013, 60,400 kilograms of cannabis were produced legally. In 2014 there were an estimated 182.5 million cannabis users (3.8% of the population aged 15-64). This percentage has not changed significantly between 1998 and 2014. Cannabis can be used by smoking, vaporizing, within food, or as an extract.

Medical cannabis (or medical marijuana) refers to the use of cannabis and its constituent cannabinoids, to treat disease or improve symptoms. Medical cannabis use takes place in Canada, Belgium, Australia, the Netherlands, Germany, Spain, and 31 U.S. states. In September 2018, cannabis was legalized in South Africa while Canada legalized recreational use of cannabis in October 2018.

Cannabis is used to reduce nausea and vomiting during chemotherapy, to improve appetite in people with HIV/AIDS, and to treat chronic pain and muscle spasms. Cannabinoids are under preliminary research for their potential to affect stroke. Short-term use increases both minor and major adverse effects. Common side effects include dizziness, feeling tired, vomiting, and hallucinations. Long-term effects of cannabis are not clear. Concerns include memory and cognition problems, risk of addiction, schizophrenia in young people, and the risk of children taking it by accident.

The main psychoactive part of cannabis is tetrahydrocannabinol (THC), one of 483 known compounds in the plant, including at least 65 other cannabinoids. Cannabis has mental and physical effects, such as creating a “high” or “stoned” feeling, a general change in perception, heightened mood, and an increase in appetite. Onset of effects is within minutes when smoked, and about 30 to 60 minutes when cooked and eaten. They last for between two and six hours. The high lipid-solubility of cannabinoids results in their persisting in the body for long periods of time. Even after a single administration of THC, detectable levels of THC can be found in the body for weeks or longer (depending on the amount administered and the sensitivity of the assessment method). A number of investigators have suggested that this is an important factor in marijuana's effects, perhaps because cannabinoids may accumulate in the body, particularly in the lipid membranes of neurons.

Researchers have confirmed that THC exerts its most prominent effects via its actions on two types of cannabinoid receptors, the CB₁ receptor and the CB₂ receptor, both of which are G protein-coupled receptors. The CB₁ receptor is found primarily in the brain as well as in some peripheral tissues, and the CB₂ receptor is found primarily in peripheral tissues but is also expressed in neuroglial cells. THC appears to alter mood and cognition through its agonist actions on the CB₁ receptors, which inhibit a secondary messenger system (adenylate cyclase) in a dose-dependent manner. These actions can be blocked by the selective CB₁ receptor antagonist rimonabant (SR141716), which has been shown in clinical trials to be an effective treatment for smoking cessation, weight loss, and as a means of controlling or reducing metabolic syndrome risk factors. However, due to the dysphoric effect of CB₁ receptor antagonists, this drug is often discontinued due to these side effects.

Via CB₁ receptor activation, THC indirectly increases dopamine release and produces psychotropic effects. Cannabidiol (CBD) also acts as an allosteric modulator of the μ- and δ-opioid receptors. THC also potentiates the effects of the glycine receptors. It is unknown if or how these actions contribute to the effects of cannabis. CBD is a 5-HT_{1A} receptor agonist, which may also contribute to an anxiolytic effect. This likely means the high concentrations of CBD found in Cannabis indica mitigate the anxiogenic effect of THC significantly. The cannabis industry claims that sativa strains provide a more stimulating psychoactive high while indica strains are more sedating with a body high; however, this is disputed by researchers.

2. Synthetic Materials

Synthetic cannabinoids are a class of molecules that bind to cannabinoid receptors in the body (the same receptors to which THC and CBD attach, which are cannabinoids in cannabis plants). They are designer drugs that are commonly sprayed onto plant matter and are usually smoked, although since 2016 they have also been consumed in a concentrated liquid form in the US and UK. They have been marketed as herbal incense, or “herbal smoking blends.” They are often labeled “not for human consumption” for liability defense.

When the herbal blends went on sale in the early 2000s, it was thought that they achieved psychoactive effects from a mixture of natural herbs. Laboratory analysis in 2008 showed instead that many contained synthetic cannabinoids. Since 2016 synthetic cannabinoids are the most common new psychoactive substances to be reported. From 2008 to 2014, 142 synthetic cannabinoids were reported to the European Monitoring Centre for Drugs and Drug Addiction (EMCDDA). A large and complex variety of synthetic cannabinoids are designed in an attempt to avoid legal restrictions on cannabis, making synthetic cannabinoids designer drugs.

Most synthetic cannabinoids are agonists of the cannabinoid receptors. They have been designed to be similar to THC, the natural cannabinoid with the strongest binding affinity to the CB₁ receptor, which is linked to the psychoactive effects or “high” of marijuana. These synthetic analogs often have greater binding affinity and greater potency to the CB₁ receptors. There are several synthetic cannabinoid families (e.g., CP-xxx, WIN-xxx, JWH-xxx, UR-xxx, and PB-xx) classified based on the base structure.

Reported user negative effects include palpitations, paranoia, intense anxiety, nausea, vomiting, confusion, poor coordination, and seizures. There have also been reports of a strong compulsion to re-dose, withdrawal symptoms, and persistent cravings. There have been several deaths linked to synthetic cannabinoids. The Centers for Disease Control and Prevention (CDC) found that the number of deaths from synthetic cannabinoid use tripled between 2014 and 2015.

Use of the term “synthetic marijuana” to describe products containing synthetic cannabinoids is controversial and a misnomer. Relative to marijuana, is has been argued that products containing synthetic cannabinoids are quite different, and the effects are much more unpredictable. Since the term synthetic does not apply to the plant, but rather to the cannabinoid that the plant contains (THC), the term synthetic cannabinoid is more appropriate.

Synthetic cannabinoids were made for cannabinoid research focusing on tetrahydrocannabinol (THC), the main psychoactive and analgesic compound found in the cannabis plant. Synthetic cannabinoids were needed partly due to legal restrictions on natural cannabinoids, which make them difficult to obtain for research. Tritium-labelled cannabinoids such as CP-55,940 were instrumental in discovering the cannabinoid receptors in the early 1990s.

Some early synthetic cannabinoids were also used clinically. Nabilone, a first-generation synthetic THC analog, has been used as an antiemetic to combat vomiting and nausea, since 1981. Synthetic THC (marinol, dronabinol) has been used as an antiemetic since 1985 and an appetite stimulant since 1991.

In the early 2000s, synthetic cannabinoids began to be used for recreational drug use in an attempt to get similar effects to cannabis. Because synthetic cannabinoid molecular structures differ from THC and other illegal cannabinoids, synthetic cannabinoids were not technically illegal. Since the discovery of the use of synthetic cannabinoids for recreational use in 2008, some synthetic cannabinoids have been made illegal, but new analogs are continually synthesized to avoid the restrictions. Synthetic cannabinoids have also been used recreationally because they are inexpensive and are typically not revealed by the standard marijuana drug tests. Unlike nabilone, the synthetic cannabinoids found being used for recreational use did not have any documented therapeutic effects.

There are five major categories for synthetic cannabinoids: classical cannabinoids, non-classical cannabinoids, hybrid cannabinoids, aminoalkylindoles, and eicosanoids. Classical cannabinoids are analogs of THC that are based on a dibenzopyran ring. They were developed starting in the 1960s, following the isolation of THC, and were originally the only cannabinoids synthesized. Classical cannabinoids include nabilone and dronabinol, and one of the best known synthetic classical cannabinoids is HU-210. HU-210 is a chiral compound first synthesized by Raphael Mechoulam at Hebrew University in the 1980s.

Non-classical cannabinoids include cyclohexylphenols (CP), which were first synthesized in the late 1970s to 1980s by Pfizer as potential analgesics. The C8 homologue of CP-47,497 (CP-47,497-C8) was one of the first synthetic cannabinoids being used recreationally. CP-47,497-C8 is made by extending the dimethylheptyl side chain of CP-47,497 to a dimethyloctyl side chain. It was discovered by forensic scientists in an herbal blend known as “Spice” in 2008, along with JWH-018, an aminoalkylindole.

Hybrid cannabinoids have a combination of classical and non-classical cannabinoid structural features. For example, AM-4030, a derivative of HU-210, is a hybrid cannabinoid because it has the dibenzopyran ring common of classical cannabinoids and an aliphatic hydroxyl group common in the CP family of nonclassical cannabinoids.

Aminoalkylindoles are structurally dissimilar to THC and include naphthoylindoles (JWH-018), phenylacetylindoles (JWH-250), and benzoylindoles (AM-2233). Aminoalkylindoles are considered to be the most common synthetic cannabinoids found in synthetic cannabinoid blends, likely due to the fact that these molecules are easier to synthesize than classical and non-classical cannabinoids. The JWH molecules were first synthesized by Professor John William Huffman at Clemson University in the late 1990s. The FBI concluded in a 2012 memo that as a result of the publication of J. W. Huffman's research, people searching for a “marijuana-like-high” would follow his recipes and methods.

Eicosanoid synthetic cannabinoids are analogs of endocannabinoids, such as anandamide. Endocannabinoids are cannabinoids naturally occurring in the body. One of the best-known synthetic analogs of anandamide is methanandamide.

The synthetic cannabinoids that have emerged recently have even greater structural diversity, possibly to subvert legal regulations on earlier generations of synthetic cannabinoids. The indazole carboxamide group, including APINACA (AKB-48), an adamantyl indazole carboxamide, and AB-PINACA, an aminocarbonyl indazole carboxamide, is an example of a new group of synthetic cannabinoids. Most clandestine manufacturers and producers only make small changes to the structure of a synthetic cannabinoid, such as changing an indole to indazole structure (AM-2201 to THJ-2201) or terminal fluorine replacement; however, one group that was unprecedented when discovered by forensic scientists in 2013, was the quinolinyl ester synthetic cannabinoids.

PB-22 and 5F-PB-22 were the first synthetic cannabinoids to include a quinoline substructure and an ester linkage. These compounds are thought to have been synthesized with the intention of making a synthetic cannabinoid prodrug, which might improve absorption and confound detection. Ester bonds are easily biodegradable through spontaneous or endogenous, nonspecific esterase hydrolysis, which has been commonly used in medicinal chemistry to make ester prodrugs.

Although most synthetic cannabinoids are not direct analogs of THC, they share many common features with THC. Most are lipid-soluble, non-polar, small molecules (usually 20-26 carbon atoms) that are fairly volatile, making them “smokable,” like THC. Another common feature of most synthetic cannabinoids and THC is a side chain of 5-9 saturated carbon atoms. It has been found that this chain of 5-9 carbons is required for optimal psychotropic activity from binding CB₁ receptors. Also, most synthetic cannabinoids are agonists of both cannabinoid receptors, CB₁ and CB₂, like THC; however, they often have greater binding affinity and therefore greater potency than THC, as seen in Table 2. Due to the greater potency, the standard doses of many synthetic cannabinoids may be less than 1 mg.

### VI. Treating Diseases and Disorders

The method of treatment comprises providing to a patient in need of treatment a dry powder inhaler comprising a cartridge containing a dose of an inhalable formulation comprising a cannabis agent and a pharmaceutical acceptable carrier and/or excipient; and having the patient inhale through the dry powder inhaler deeply for about 3 to 4 seconds to deliver the dose. In the method, the patient can resume normal breathing pattern thereafter.

Medical cannabis has several potential beneficial effects. Evidence is moderate that it helps in chronic pain and muscle spasms. Other evidence suggests its use for reducing nausea during chemotherapy, improving appetite in HIV/AIDS, improving sleep, and improving tics in Tourette syndrome. When usual treatments are ineffective, cannabinoids have also been recommended for anorexia, arthritis, migraine, and glaucoma. It is recommended that cannabis use be stopped in pregnancy.

1. Nausea

Medical cannabis is somewhat effective in chemotherapy-induced nausea and vomiting (CINV) and may be a reasonable option in those who do not improve following preferential treatment. Comparative studies have found cannabinoids to be more effective than some conventional antiemetics such as prochlorperazine, promethazine, and metoclopramide in controlling CINV, but these are used less frequently because of side effects including dizziness, dysphoria, and hallucinations. Long-term cannabis use may cause nausea and vomiting, a condition known as cannabinoid hyperemesis syndrome.

A 2016 Cochrane review said that cannabinoids were “probably effective” in treating chemotherapy-induced nausea in children, but with a high side-effect profile (mainly drowsiness, dizziness, altered moods, and increased appetite). Less common side effects were ocular problems, orthostatic hypotension, muscle twitching, pruritis, vagueness, hallucinations, lightheadedness and dry mouth.

2. HIV/AIDS

Evidence is lacking for both efficacy and safety of cannabis and cannabinoids in treating patients with HIV/AIDS or for anorexia associated with AIDS. As of 2013, current studies suffer from effects of bias, small sample size, and lack of long-term data.

3. Pain

A 2017 review found only limited evidence for the effectiveness of cannabis in relieving chronic pain in several conditions. Another review found tentative evidence for use of cannabis in treating peripheral neuropathy, but little evidence of benefit for other types of long-term pain.

When cannabis is inhaled to relieve pain, blood levels of cannabinoids rise faster than when oral products are used, peaking within three minutes and attaining an analgesic effect in seven minutes. A 2014 review found limited and weak evidence that smoked cannabis was effective for chronic non-cancer pain. A 2015 meta-analysis found that inhaled medical cannabis was effective in reducing neuropathic pain in the short term for one in five to six patients. Another 2015 review found limited evidence that medical cannabis was effective for neuropathic pain when combined with traditional analgesics. A 2011 review considered cannabis to be generally safe, and it appears safer than opioids in palliative care.

4. Neurological Problems

Cannabis' efficacy is not clear in treating neurological problems, including multiple sclerosis (MS), epilepsy, and movement problems. The combination of 49-tetrahydrocannabinol (THC) and cannabidiol (CBD) extracts give subjective relief of spasticity, though objective post-treatment assessments do not reveal significant changes. Evidence also suggests that oral cannabis extract is effective for reducing patient-centered measures of spasticity. A trial of cannabis is deemed to be a reasonable option if other treatments have not been effective. Its use for MS is approved in ten countries. A 2012 review found no problems with tolerance, abuse, or addiction.

5. Epilepsy

Epilepsy (also called epileptic seizure disorder) is a chronic brain disorder characterized by recurrent (≥2) seizures that are unprovoked (i.e., not related to reversible stressors) and that occur >24 h apart. A single seizure is not considered an epileptic seizure. Epilepsy is often idiopathic, but various brain disorders, such as malformations, strokes, and tumors, can cause symptomatic epilepsy.

6. Dravet Syndrome

Dravet syndrome is a severe infantile-onset, genetic, drug-resistant epilepsy syndrome with a distinctive but complex electroclinical presentation. Onset of Dravet syndrome occurs during the first year of life with clonic seizures (jerking) and tonic-clonic (convulsive) seizures in previously healthy and developmentally normal infants. Symptoms peak at about five months of age, and the latest onset beginning by 15 months of age. Other seizures develop between one and four years of age such as prolonged focal dyscognitive seizures and brief absence seizures, and duration of these seizures decreases during this period, but their frequency increases. Prognosis is poor, with death occurring in approximately 14 percent of children. Death can be caused by the seizures themselves, by infection due to prolonged periods of physical inactivity, or by the presence of advanced neurodegenerative disease or a compromised level of consciousness requiring a feeding tube. Death can also occur suddenly due to uncertain causes, often because of the relentless neurological decline or from Sudden Unexpected Death in Epilepsy.

7. Lennox-Gastaut Syndrome (LGS)

LGS is a type of epilepsy with multiple types of seizures, particularly tonic (stiffening) and atonic (drop) seizures. According to Trevathan et al. in the December 1997 edition of Epilepsia, the estimated prevalence of LGS is between 3 percent and 4 percent of childhood epilepsy cases. LGS affects between 14,500 to 18,500 children under the age of 18 years in the U.S. and over 30,000 children and adults in the U.S. Eighty percent of children with LGS continue to experience seizures, psychiatric, intellectual and behavioral deficits in adulthood. Seizures due to LGS are hard to control and generally require life-long treatment. Also, Epidiolex® (a cannabidiol oral solution) is used for treatment of seizures associated with Dravet syndrome and LGS.

8. Tuberous Sclerosis Complex

Tuberous sclerosis complex (TSC) is a neurocutaneous syndrome that occurs in 1 of 6000 children; 85% of cases involve mutations in the TSC1 gene (904), which controls the production of hamartin, or the TSC2 gene (16p13.3), which controls the production of tuberin. These proteins act as growth suppressors. If either parent has the disorder, children have a 50% risk of having it. However, new mutations account for two thirds of cases. Patients with TSC have tumors or abnormalities that manifest at different ages and in multiple organs, including the brain, heart, eyes, kidneys, lungs and skin

9. Rett Syndrome

Rett syndrome (RTT) is a rare, non-inherited, X-linked neurodevelopmental disorder affecting approximately 1 in 10,000 to 15,000 live female births. RTT is most commonly caused by heterozygous de-novo mutations in the gene encoding methyl-CpG-binding protein 2 (MeCP2) resulting in a loss of function of the MeCP2 protein. The condition affects predominantly females and it results in abnormal neuronal development and function in affected children. The symptomatology of RTT is progressive, with early onset from about 6-18 months of life, followed by a rapid destructive phase at the age of 1 to 4 years. This stage is characterized by loss of purposeful hand skills, loss of spoken language, breathing and cardiac irregularities, microcephaly, and autistic-like behaviors. After the period of regression, patients enter a prolonged period of stabilization where most of the impairments associated with the destructive phase persist together with apraxia, motor problems, and seizures. Over time, the patient's motor function continues to deteriorate, resulting in reduced mobility, scoliosis, rigidity, muscular weakness and spasticity.

10. Autism Spectrum Disorders

Autism spectrum disorders are neurodevelopmental disorders characterized by impaired social interaction and communication, repetitive and stereotyped patterns of behavior, and uneven intellectual development often with intellectual disability. Symptoms begin in early childhood. The cause in most children is unknown, although evidence supports a genetic component; in some patients, the disorders may be caused by a medical condition. Diagnosis is based on developmental history and observation. Treatment consists of behavioral management and sometimes drug therapy. Autism spectrum disorders represent a range of neurodevelopmental differences that are considered neurodevelopmental disorders.

11. Post-Traumatic Stress Disorder

There is tentative evidence that medical cannabis is effective at reducing posttraumatic stress disorder symptoms, but, as of 2017, there is insufficient evidence to confirm its effectiveness for this condition.

12. Neurodegenrative Diseases

A. Parkinson's Disease

Parkinson's disease (PD) is a long-term degenerative disorder of the central nervous system that mainly affects the motor system. The symptoms generally come on slowly over time. Early in the disease, the most obvious are shaking, rigidity, slowness of movement, and difficulty with walking. Thinking and behavioral problems may also occur. Dementia becomes common in the advanced stages of the disease. Depression and anxiety are also common occurring in more than a third of people with PD. Other symptoms include sensory, sleep, and emotional problems. The main motor symptoms are collectively called “parkinsonism” or a “parkinsonian syndrome.”

The cause of Parkinson's disease is generally unknown but believed to involve both genetic and environmental factors. Those with a family member affected are more likely to get the disease themselves. There is also an increased risk in people exposed to certain pesticides and among those who have had prior head injuries while there is a reduced risk in tobacco smokers and those who drink coffee or tea. The motor symptoms of the disease result from the death of cells in the substantia nigra, a region of the midbrain. This results in not enough dopamine in these areas. The reason for this cell death is poorly understood but involves the build-up of proteins into Lewy bodies in the neurons. Diagnosis of typical cases is mainly based on symptoms, with tests such as neuroimaging being used to rule out other diseases.

There is no cure for Parkinson's disease. Initial treatment is typically with the anti-parkinson medication L-DOPA (levodopa), with dopamine agonists being used once levodopa becomes less effective. As the disease progresses and neurons continue to be lost, these medications become less effective while at the same time they produce a complication marked by involuntary writhing movements. Diet and some forms of rehabilitation have shown some effectiveness at improving symptoms. Surgery to place microelectrodes for deep brain stimulation has been used to reduce motor symptoms in severe cases where drugs are ineffective. Evidence for treatments for the non-movement-related symptoms of PD, such as sleep disturbances and emotional problems, is less strong.

B. Huntington's Disease

Huntington's disease. Huntington's disease (HD), also known as Huntington's chorea, is an inherited disorder that results in death of brain cells. The earliest symptoms are often subtle problems with mood or mental abilities. A general lack of coordination and an unsteady gait often follow. As the disease advances, uncoordinated, jerky body movements become more apparent. Physical abilities gradually worsen until coordinated movement becomes difficult and the person is unable to talk. Mental abilities generally decline into dementia. The specific symptoms vary somewhat between people. Symptoms usually begin between 30 and 50 years of age but can start at any age. The disease may develop earlier in life in each successive generation. About 8% of cases start before the age of 20 years and typically present with symptoms more similar to Parkinson's disease. People with HD often underestimate the degree of their problems.

HD is typically inherited from a person's parents with 10% of cases due to a new mutation. The disease is caused by an autosomal dominant mutation in either of an individual's two copies of a gene called Huntingtin. This means a child of an affected person typically has a 50% chance of inheriting the disease. The Huntingtin gene provides the genetic information for a protein that is also called “huntingtin.” Expansion of CAG (cytosine-adenine-guanine) triplet repeats in the gene coding for the Huntingtin protein results in an abnormal protein, which gradually damages cells in the brain, through mechanisms that are not fully understood. Diagnosis is by genetic testing, which can occur at any point in time regardless of whether or not symptoms are present. This fact raises several ethical debates: the age at which an individual is considered mature enough to choose testing; whether parents have the right to have their children tested; and managing confidentiality and disclosure of test results.

There is no cure for HD. Full-time care is required in the later stages of the disease. Treatments can relieve some symptoms and in some improve quality of life. The best evidence for treatment of the movement problems is with tetrabenazine. HD affects about 4 to 15 in 100,000 people of European descent. It is rare among Japanese and occurs at an unknown rate in Africa. The disease affects men and women equally. Complications such as pneumonia, heart disease, and physical injury from falls reduce life expectancy. Suicide is the cause of death in about 9% of cases. Death typically occurs fifteen to twenty years from when the disease was first detected.

Symptoms of Huntington's disease most commonly become noticeable between the ages of 35 and 44 years, but they can begin at any age from infancy to old age. In the early stages, there are subtle changes in personality, cognition, and physical skills. The physical symptoms are usually the first to be noticed, as cognitive and behavioral symptoms are generally not severe enough to be recognized on their own at the earlier stages. Almost everyone with Huntington's disease eventually exhibits similar physical symptoms, but the onset, progression and extent of cognitive and behavioral symptoms vary significantly between individuals.

The most characteristic initial physical symptoms are jerky, random, and uncontrollable movements called chorea. Chorea may be initially exhibited as general restlessness, small unintentionally initiated or uncompleted motions, lack of coordination, or slowed saccadic eye movements. These minor motor abnormalities usually precede more obvious signs of motor dysfunction by at least three years. The clear appearance of symptoms such as rigidity, writhing motions or abnormal posturing appear as the disorder progresses. These are signs that the system in the brain that is responsible for movement has been affected. Psychomotor functions become increasingly impaired, such that any action that requires muscle control is affected. Common consequences are physical instability, abnormal facial expression, and difficulties chewing, swallowing, and speaking. Eating difficulties commonly cause weight loss and may lead to malnutrition. Sleep disturbances are also associated symptoms. Juvenile HD differs from these symptoms in that it generally progresses faster and chorea is exhibited briefly, if at all, with rigidity being the dominant symptom. Seizures are also a common symptom of this form of HD.

Cognitive abilities are progressively impaired. Especially affected are executive functions which include planning, cognitive flexibility, abstract thinking, rule acquisition, initiation of appropriate actions, and inhibition of inappropriate actions. As the disease progresses, memory deficits tend to appear. Reported impairments range from short-term memory deficits to long-term memory difficulties, including deficits in episodic (memory of one's life), procedural (memory of the body of how to perform an activity) and working memory. Cognitive problems tend to worsen over time, ultimately leading to dementia. This pattern of deficits has been called a subcortical dementia syndrome to distinguish it from the typical effects of cortical dementias, e.g., Alzheimer's disease.

Reported neuropsychiatric manifestations are anxiety, depression, a reduced display of emotions (blunted affect), egocentrism, aggression, and compulsive behavior, the latter of which can cause or worsen addictions, including alcoholism, gambling, and hypersexuality. Difficulties in recognizing other people's negative expressions have also been observed. The prevalence of these symptoms is highly variable between studies, with estimated rates for lifetime prevalence of psychiatric disorders between 33% and 76%. For many sufferers and their families, these symptoms are among the most distressing aspects of the disease, often affecting daily functioning and constituting reason for institutionalization. Suicidal thoughts and suicide attempts are more common than in the general population. Often individuals have reduced awareness of chorea, cognitive and emotional impairments.

Mutant Huntingtin is expressed throughout the body and associated with abnormalities in peripheral tissues that are directly caused by such expression outside the brain. These abnormalities include muscle atrophy, cardiac failure, impaired glucose tolerance, weight loss, osteoporosis, and testicular atrophy.

All humans have two copies of the Huntingtin gene (HTT), which codes for the protein Huntingtin (HTT). The gene is also called HD and IT15, which stands for ‘interesting transcript 15’. Part of this gene is a repeated section called a trinucleotide repeat, which varies in length between individuals and may change length between generations. If the repeat is present in a healthy gene, a dynamic mutation may increase the repeat count and result in a defective gene. When the length of this repeated section reaches a certain threshold, it produces an altered form of the protein, called mutant Huntingtin protein (mHTT). The differing functions of these proteins are the cause of pathological changes which in turn cause the disease symptoms. The Huntington's disease mutation is genetically dominant and almost fully penetrant: mutation of either of a person's HTT alleles causes the disease. It is not inherited according to sex, but the length of the repeated section of the gene and hence its severity can be influenced by the sex of the affected parent.

HD is one of several trinucleotide-repeat disorders which are caused by the length of a repeated section of a gene exceeding a normal range. The HTT gene is located on the short arm of chromosome 4 at 4p16.3. HTT contains a sequence of three DNA bases—cytosine-adenine-guanine (CAG)—repeated multiple times (i.e., . . . CAGCAGCAG . . . ), known as a trinucleotide repeat. CAG is the 3-letter genetic code (codon) for the amino acid glutamine, so a series of them results in the production of a chain of glutamine known as a polyglutamine tract (or polyQ tract), and the repeated part of the gene, the PolyQ region.

Generally, people have fewer than 36 repeated glutamines in the polyQ region which results in production of the cytoplasmic protein Huntingtin. However, a sequence of 36 or more glutamines results in the production of a protein which has different characteristics. This altered form, called mutant huntingtin (mHTT), increases the decay rate of certain types of neurons. Regions of the brain have differing amounts and reliance on these types of neurons and are affected accordingly. Generally, the number of CAG repeats is related to how much this process is affected, and accounts for about 60% of the variation of the age of the onset of symptoms. The remaining variation is attributed to environment and other genes that modify the mechanism of HD. 36-39 repeats result in a reduced-penetrance form of the disease, with a much later onset and slower progression of symptoms. In some cases, the onset may be so late that symptoms are never noticed. With very large repeat counts, HD has full penetrance and can occur under the age of 20, when it is then referred to as juvenile HD, akinetic-rigid, or Westphal variant HD. This accounts for about 7% of HD carriers.

13. Schizophrenia

Schizophrenia is a mental illness characterized by abnormal behavior, strange speech, and a decreased ability to understand reality. Other symptoms may include false beliefs, unclear or confused thinking, hearing voices that do not exist, reduced social engagement and emotional expression, and lack of motivation. People with schizophrenia often have additional mental health problems such as anxiety, depression, or substance-use disorders. Symptoms typically come on gradually, begin in young adulthood, and, in many cases, never resolve.

The causes of schizophrenia include environmental and genetic factors. Possible environmental factors include being raised in a city, cannabis use during adolescence, certain infections, the age of a person's parents, and poor nutrition during pregnancy. Genetic factors include a variety of common and rare genetic variants. Diagnosis is based on observed behavior, the person's reported experiences and reports of others familiar with the person. During diagnosis, a person's culture must also be taken into account. As of 2013, there is no objective test. Schizophrenia does not imply a “split personality” or dissociative identity disorder, conditions with which it is often confused in public perception.

The mainstay of treatment is antipsychotic medication, along with counselling, job training, and social rehabilitation. It is unclear whether typical or atypical antipsychotics are better. In those who do not improve with other antipsychotics, clozapine may be tried. In more serious situations where there is risk to self or others, involuntary hospitalization may be necessary, although hospital stays are now shorter and less frequent than they once were.

About 0.3% to 0.7% of people are affected by schizophrenia during their lifetimes. In 2013, there were an estimated 23.6 million cases globally. Males are more often affected, and onset is on average earlier in age. About 20% of people eventually do well, and a few recover completely. About 50% have lifelong impairment. Social problems, such as long-term unemployment, poverty, and homelessness, are common. The average life expectancy of people with the disorder is 10-25 years less than that of the general population. This is the result of increased physical health problems and a higher suicide rate (about 5%). In 2015, an estimated 17,000 people worldwide died from behavior related to, or caused by, schizophrenia.

14. Stroke

Stroke is a medical condition in which poor blood flow to the brain results in cell death. There are two main types of stroke: ischemic, due to lack of blood flow, and hemorrhagic, due to bleeding. They result in part of the brain not functioning properly. Signs and symptoms of a stroke may include an inability to move or feel on one side of the body, problems understanding or speaking, feeling like the world is spinning, or loss of vision to one side. Signs and symptoms often appear soon after the stroke has occurred. If symptoms last less than one or two hours, it is known as a transient ischemic attack (TIA) or mini-stroke. A hemorrhagic stroke may also be associated with a severe headache. The symptoms of a stroke can be permanent. Long-term complications may include pneumonia or loss of bladder control.

The main risk factor for stroke is high blood pressure. Other risk factors include tobacco smoking, obesity, high blood cholesterol, diabetes mellitus, previous TIA, and atrial fibrillation. An ischemic stroke is typically caused by blockage of a blood vessel, though there are also less common causes. A hemorrhagic stroke is caused by either bleeding directly into the brain or into the space between the brain's membranes. Bleeding may occur due to a ruptured brain aneurysm. Diagnosis is typically with medical imaging such as a CT scan or magnetic resonance imaging (MRI) scan along with a physical exam. Other tests such as an electrocardiogram (ECG) and blood tests are done to determine risk factors and rule out other possible causes. Low blood sugar may cause similar symptoms.

Prevention includes decreasing risk factors, as well as possibly aspirin, statins, surgery to open up the arteries to the brain in those with problematic narrowing, and warfarin in those with atrial fibrillation. A stroke or TIA often requires emergency care. An ischemic stroke, if detected within three to four and half hours, may be treatable with a medication that can break down the clot. Aspirin should be used. Some hemorrhagic strokes benefit from surgery. Treatment to try to recover lost function is called stroke rehabilitation and ideally takes place in a stroke unit; however, these are not available in much of the world.

Strokes can be classified into two major categories: ischemic and hemorrhagic. Ischemic strokes are caused by interruption of the blood supply to the brain, while hemorrhagic strokes result from the rupture of a blood vessel or an abnormal vascular structure. About 87% of strokes are ischemic, the rest being hemorrhagic. Bleeding can develop inside areas of ischemia, a condition known as “hemorrhagic transformation.” It is unknown how many hemorrhagic strokes actually start as ischemic strokes.

Definition. In the 1970s the World Health Organization defined stroke as a “neurological deficit of cerebrovascular cause that persists beyond 24 hours or is interrupted by death within 24 hours”, although the word “stroke” is centuries old. This definition was supposed to reflect the reversibility of tissue damage and was devised for the purpose, with the time frame of 24 hours being chosen arbitrarily. The 24-hour limit divides stroke from transient ischemic attack, which is a related syndrome of stroke symptoms that resolve completely within 24 hours. With the availability of treatments which can reduce stroke severity when given early, many now prefer alternative terminology, such as brain attack and acute ischemic cerebrovascular syndrome (modeled after heart attack and acute coronary syndrome, respectively), to reflect the urgency of stroke symptoms and the need to act swiftly.

In an ischemic stroke, blood supply to part of the brain is decreased, leading to dysfunction of the brain tissue in that area. There are four reasons why this might happen: thrombosis (obstruction of a blood vessel by a blood clot forming locally), embolism (obstruction due to an embolus from elsewhere in the body, see below), systemic hypoperfusion (general decrease in blood supply, e.g., in shock), or cerebral venous sinus thrombosis. Stroke without an obvious explanation is termed “cryptogenic” (of unknown origin); this constitutes 30-40% of all ischemic strokes.

There are various classification systems for acute ischemic stroke. The Oxford Community Stroke Project classification (OCSP, also known as the Bamford or Oxford classification) relies primarily on the initial symptoms; based on the extent of the symptoms, the stroke episode is classified as total anterior circulation infarct (TACI), partial anterior circulation infarct (PACI), lacunar infarct (LACI) or posterior circulation infarct (POCI). These four entities predict the extent of the stroke, the area of the brain that is affected, the underlying cause, and the prognosis. The TOAST (Trial of Org 10172 in Acute Stroke Treatment) classification is based on clinical symptoms as well as results of further investigations; on this basis, a stroke is classified as being due to (1) thrombosis or embolism due to atherosclerosis of a large artery, (2) an embolism originating in the heart, (3) complete blockage of a small blood vessel, (4) other determined cause, (5) undetermined cause (two possible causes, no cause identified, or incomplete investigation). Users of stimulants, such as cocaine and methamphetamine are at a high risk for ischemic strokes.

There are two main types of hemorrhagic stroke: intracerebral hemorrhage, which is basically bleeding within the brain itself (when an artery in the brain bursts, flooding the surrounding tissue with blood), due to either intraparenchymal hemorrhage (bleeding within the brain tissue) or intraventricular hemorrhage (bleeding within the brain's ventricular system); and subarachnoid hemorrhage, which is basically bleeding that occurs outside of the brain tissue but still within the skull, and precisely between the arachnoid mater and pia mater (the delicate innermost layer of the three layers of the meninges that surround the brain).

The above two main types of hemorrhagic stroke are also two different forms of intracranial hemorrhage, which is the accumulation of blood anywhere within the cranial vault; but the other forms of intracranial hemorrhage, such as epidural hematoma (bleeding between the skull and the dura mater, which is the thick outermost layer of the meninges that surround the brain) and subdural hematoma (bleeding in the subdural space), are not considered “hemorrhagic strokes.”

Hemorrhagic strokes may occur on the background of alterations to the blood vessels in the brain, such as cerebral amyloid angiopathy, cerebral arteriovenous malformation and an intracranial aneurysm, which can cause intraparenchymal or subarachnoid hemorrhage.

In addition to neurological impairment, hemorrhagic strokes usually cause specific symptoms (for instance, subarachnoid hemorrhage classically causes a severe headache known as a thunderclap headache) or reveal evidence of a previous head injury.

Signs and symptoms. Stroke symptoms typically start suddenly, over seconds to minutes, and in most cases do not progress further. The symptoms depend on the area of the brain affected. The more extensive the area of the brain affected, the more functions that are likely to be lost. Some forms of stroke can cause additional symptoms. For example, in intracranial hemorrhage, the affected area may compress other structures. Most forms of stroke are not associated with a headache, apart from subarachnoid hemorrhage and cerebral venous thrombosis and occasionally intracerebral hemorrhage.

Early recognition. Various systems have been proposed to increase recognition of stroke. Different findings are able to predict the presence or absence of stroke to different degrees. Sudden-onset face weakness, arm drift (i.e., if a person, when asked to raise both arms, involuntarily lets one arm drift downward) and abnormal speech are the findings most likely to lead to the correct identification of a case of stroke increasing the likelihood by 5.5 when at least one of these is present). Similarly, when all three of these are absent, the likelihood of stroke is significantly decreased (likelihood ratio of 0.39). While these findings are not perfect for diagnosing stroke, the fact that they can be evaluated relatively rapidly and easily make them very valuable in the acute setting.

A mnemonic to remember the warning signs of stroke is FAST (facial droop, arm weakness, speech difficulty, and time to call emergency services), as advocated by the Department of Health (United Kingdom) and the Stroke Association, the American Stroke Association, the National Stroke Association (US), the Los Angeles Prehospital Stroke Screen (LAPSS) and the Cincinnati Prehospital Stroke Scale (CPSS). Use of these scales is recommended by professional guidelines.

For people referred to the emergency room, early recognition of stroke is deemed important as this can expedite diagnostic tests and treatments. A scoring system called ROSIER (recognition of stroke in the emergency room) is recommended for this purpose; it is based on features from the medical history and physical examination.

15. Depression

Major depressive disorder (MDD), also known simply as depression, is a mental disorder characterized by at least two weeks of low mood that is present across most situations. It is often accompanied by low self-esteem, loss of interest in normally enjoyable activities, low energy, and pain without a clear cause. People may also occasionally have false beliefs or see or hear things that others cannot. Some people have periods of depression separated by years in which they are normal, while others nearly always have symptoms present. Major depressive disorder can negatively affect a person's personal life, work life, or education, as well as sleeping, eating habits, and general health. Between 2-8% of adults with major depression die by suicide, and about 50% of people who die by suicide had depression or another mood disorder.

The cause is believed to be a combination of genetic, environmental, and psychological factors. Risk factors include a family history of the condition, major life changes, certain medications, chronic health problems, and substance abuse. About 40% of the risk appears to be related to genetics. The diagnosis of major depressive disorder is based on the person's reported experiences and a mental status examination. There is no laboratory test for major depression. Testing, however, may be done to rule out physical conditions that can cause similar symptoms. Major depression is more severe and lasts longer than sadness, which is a normal part of life.

Typically, people are treated with counseling and antidepressant medication. Medication appears to be effective, but the effect may only be significant in the most severely depressed. It is unclear whether medications affect the risk of suicide. Types of counseling used include cognitive behavioral therapy (CBT) and interpersonal therapy. If other measures are not effective, electroconvulsive therapy (ECT) may be considered. Hospitalization may be necessary in cases with a risk of harm to self and may occasionally occur against a person's wishes.

Major depressive disorder affected approximately 216 million people (3% of the world's population) in 2015. The percentage of people who are affected at one point in their life varies from 7% in Japan to 21% in France. Lifetime rates are higher in the developed world (15%) compared to the developing world (11%). It causes the second-most years lived with disability, after lower back pain. The most common time of onset is in a person's 20s and 30s. Females are affected about twice as often as males.

16. Traumatic Brain Injury

Traumatic brain injury (TBI), also known as intracranial injury, occurs when an external force injures the brain. TBI can be classified based on severity, mechanism (closed or penetrating head injury), or other features (e.g., occurring in a specific location or over a widespread area). Head injury is a broader category that may involve damage to other structures such as the scalp and skull. TBI can result in physical, cognitive, social, emotional, and behavioral symptoms, and outcome can range from complete recovery to permanent disability or death.

Causes include falls, vehicle collisions, and violence. Brain trauma occurs as a consequence of a sudden acceleration or deceleration within the cranium or by a complex combination of both movement and sudden impact. In addition to the damage caused at the moment of injury, a variety of events in the minutes to days following the injury may result in secondary injury. These processes include alterations in cerebral blood flow and the pressure within the skull. Some of the imaging techniques used for diagnosis include computed tomography and magnetic resonance imaging (MRIs).

Prevention measures include use of protective technology in vehicles, such as seat belts and sports or motorcycle helmets, as well as efforts to reduce the number of collisions, such as safety education programs and enforcement of traffic laws. Depending on the injury, treatment required may be minimal or may include interventions such as medications, emergency surgery or surgery years later. Physical therapy, speech therapy, recreation therapy, occupational therapy and vision therapy may be employed for rehabilitation. Counseling, supported employment, and community support services may also be useful.

TBI is a major cause of death and disability worldwide, especially in children and young adults. Males sustain traumatic brain injuries more frequently than do females. The 20th century saw developments in diagnosis and treatment that decreased death rates and improved outcome.

Traumatic brain injury is defined as damage to the brain resulting from external mechanical force, such as rapid acceleration or deceleration, impact, blast waves, or penetration by a projectile. Brain function is temporarily or permanently impaired and structural damage may or may not be detectable with current technology.

TBI is one of two subsets of acquired brain injury (brain damage that occur after birth); the other subset is non-traumatic brain injury, which does not involve external mechanical force (examples include stroke and infection). All traumatic brain injuries are head injuries, but the latter term may also refer to injury to other parts of the head. However, the terms head injury and brain injury are often used interchangeably. Similarly, brain injuries fall under the classification of central nervous system injuries and neurotrauma. In neuropsychology research literature, in general the term “traumatic brain injury” is used to refer to non-penetrating traumatic brain injuries.

TBI is usually classified based on severity, anatomical features of the injury, and the mechanism (the causative forces). Mechanism-related classification divides TBI into closed and penetrating head injury. A closed (also called nonpenetrating, or blunt) injury occurs when the brain is not exposed. A penetrating, or open, head injury occurs when an object pierces the skull and breaches the dura mater, the outermost membrane surrounding the brain.

Brain injuries can be classified into mild, moderate, and severe categories. The Glasgow Coma Scale (GCS), the most commonly used system for classifying TBI severity, grades a person's level of consciousness on a scale of 3-15 based on verbal, motor, and eye-opening reactions to stimuli. In general, it is agreed that a TBI with a GCS of 13 or above is mild, 9-12 is moderate, and 8 or below is severe. Similar systems exist for young children. However, the GCS grading system has limited ability to predict outcomes. Because of this, other classification systems such as the one shown in the table are also used to help determine severity. A current model developed by the Department of Defense and Department of Veterans Affairs uses all three criteria of GCS after resuscitation, duration of post-traumatic amnesia (PTA), and loss of consciousness (LOC). It also has been proposed to use changes that are visible on neuroimaging, such as swelling, focal lesions, or diffuse injury as method of classification. Grading scales also exist to classify the severity of mild TBI, commonly called concussion; these use duration of LOC, PTA, and other concussion symptoms.

Systems also exist to classify TBI by its pathological features. Lesions can be extra-axial, (occurring within the skull but outside of the brain) or intra-axial (occurring within the brain tissue). Damage from TBI can be focal or diffuse, confined to specific areas or distributed in a more general manner, respectively. However, it is common for both types of injury to exist in a given case.

Diffuse injury manifests with little apparent damage in neuroimaging studies, but lesions can be seen with microscopy techniques post-mortem, and in the early 2000s, researchers discovered that diffusion tensor imaging (DTI), a way of processing MRI images that shows white matter tracts, was an effective tool for displaying the extent of diffuse axonal injury. Types of injuries considered diffuse include edema (swelling) and diffuse axonal injury, which is widespread damage to axons including white matter tracts and projections to the cortex. Types of injuries considered diffuse include concussion and diffuse axonal injury, widespread damage to axons in areas including white matter and the cerebral hemispheres.

Focal injuries often produce symptoms related to the functions of the damaged area. Research shows that the most common areas to have focal lesions in non-penetrating traumatic brain injury are the orbitofrontal cortex (the lower surface of the frontal lobes) and the anterior temporal lobes, areas that are involved in social behavior, emotion regulation, olfaction, and decision-making, hence the common social/emotional and judgment deficits following moderate-severe TBI. Symptoms such as hemiparesis or aphasia can also occur when less commonly affected areas such as motor or language areas are, respectively, damaged.

One type of focal injury, cerebral laceration, occurs when the tissue is cut or torn. Such tearing is common in orbitofrontal cortex in particular, because of bony protrusions on the interior skull ridge above the eyes. In a similar injury, cerebral contusion (bruising of brain tissue), blood is mixed among tissue. In contrast, intracranial hemorrhage involves bleeding that is not mixed with tissue.

Hematomas, also focal lesions, are collections of blood in or around the brain that can result from hemorrhage. Intracerebral hemorrhage, with bleeding in the brain tissue itself, is an intra-axial lesion. Extra-axial lesions include epidural hematoma, subdural hematoma, subarachnoid hemorrhage, and intraventricular hemorrhage. Epidural hematoma involves bleeding into the area between the skull and the dura mater, the outermost of the three membranes surrounding the brain. In subdural hematoma, bleeding occurs between the dura and the arachnoid mater. Subarachnoid hemorrhage involves bleeding into the space between the arachnoid membrane and the pia mater. Intraventricular hemorrhage occurs when there is bleeding in the ventricles.

Symptoms are dependent on the type of TBI (diffuse or focal) and the part of the brain that is affected. Unconsciousness tends to last longer for people with injuries on the left side of the brain than for those with injuries on the right. Symptoms are also dependent on the injury's severity. With mild TBI, the patient may remain conscious or may lose consciousness for a few seconds or minutes. Other symptoms of mild TBI include headache, vomiting, nausea, lack of motor coordination, dizziness, difficulty balancing, lightheadedness, blurred vision or tired eyes, ringing in the ears, bad taste in the mouth, fatigue or lethargy, and changes in sleep patterns. Cognitive and emotional symptoms include behavioral or mood changes, confusion, and trouble with memory, concentration, attention, or thinking Mild TBI symptoms may also be present in moderate and severe injuries.

A person with a moderate or severe TBI may have a headache that does not go away, repeated vomiting or nausea, convulsions, an inability to awaken, dilation of one or both pupils, slurred speech, aphasia (word-finding difficulties), dysarthria (muscle weakness that causes disordered speech), weakness or numbness in the limbs, loss of coordination, confusion, restlessness, or agitation. Common long-term symptoms of moderate to severe TBI are changes in appropriate social behavior, deficits in social judgment, and cognitive changes, especially problems with sustained attention, processing speed, and executive functioning. Alexithymia, a deficiency in identifying, understanding, processing, and describing emotions occurs in 60.9% of individuals with TBI. Cognitive and social deficits have long-term consequences for the daily lives of people with moderate to severe TBI but can be improved with appropriate rehabilitation.

When the pressure within the skull (intracranial pressure, abbreviated ICP) rises too high, it can be deadly. Signs of increased ICP include decreasing level of consciousness, paralysis or weakness on one side of the body, and a blown pupil, one that fails to constrict in response to light or is slow to do so. Cushing's triad, a slow heart rate with high blood pressure and respiratory depression is a classic manifestation of significantly raised ICP. Anisocoria, unequal pupil size, is another sign of serious TBI. Abnormal posturing, a characteristic positioning of the limbs caused by severe diffuse injury or high ICP, is an ominous sign.

Small children with moderate to severe TBI may have some of these symptoms but have difficulty communicating them. Other signs seen in young children include persistent crying, inability to be consoled, listlessness, refusal to nurse or eat, and irritability.

The most common causes of TBI in the U.S. include violence, transportation accidents, construction, and sports. Motor bikes are major causes, increasing in significance in developing countries as other causes reduce. The estimates that between 1.6 and 3.8 million traumatic brain injuries each year are a result of sports and recreation activities in the US. In children aged two to four, falls are the most common cause of TBI, while in older children traffic accidents compete with falls for this position. TBI is the third most common injury to result from child abuse. Abuse causes 19% of cases of pediatric brain trauma, and the death rate is higher among these cases. Although men are twice as likely to have a TBI. Domestic violence is another cause of TBI, as are work-related and industrial accidents. Firearms and blast injuries from explosions are other causes of TBI, which is the leading cause of death and disability in war zones. Also, Sativex® (a THC/cannabidiol oromucosal spray solution formulated with an extract of the cannabis sativa plant) is available to treat multiple schlerosis.

17. Ocular Disease

Diseases of the eye, referred to as ocular disease, include a wide variety of ailments. Two having particular relevance here are glaucoma, macular degeneration and retinitis pigmentosa.

Glaucoma is a group of eye diseases which result in damage to the optic nerve and cause vision loss. The most common type is open-angle glaucoma with less common types including closed-angle glaucoma and normal-tension glaucoma. Open-angle glaucoma develops slowly over time and there is no pain. Peripheral vision may begin to decrease followed by central vision resulting in blindness if not treated. Closed-angle glaucoma can present gradually or suddenly. The sudden presentation may involve severe eye pain, blurred vision, mid-dilated pupil, redness of the eye, and nausea. Vision loss from glaucoma, once it has occurred, is permanent.

Risk factors for glaucoma include increased pressure in the eye, a family history of the condition, and high blood pressure. For eye pressures a value of greater than 21 mmHg or 2.8 kPa is often used with higher pressures leading to a greater risk. However, some may have high eye pressure for years and never develop damage. Conversely, optic nerve damage may occur with normal pressure, known as normal-tension glaucoma. The mechanism of open-angle glaucoma is believed to be slow exit of aqueous humor through the trabecular meshwork while in closed-angle glaucoma the iris blocks the trabecular meshwork. Diagnosis is by a dilated eye examination. Often the optic nerve shows an abnormal amount of cupping.

If treated early it is possible to slow or stop the progression of disease with medication, laser treatment, or surgery. The goal of these treatments is to decrease eye pressure. A number of different classes of glaucoma medication are available. Laser treatments may be effective in both open-angle and closed-angle glaucoma. A number of types of glaucoma surgeries may be used in people who do not respond sufficiently to other measures. Treatment of closed-angle glaucoma is a medical emergency.

About 6 to 67 million people have glaucoma globally. The disease affects about 2 million people in the United States. It occurs more commonly among older people. Closed-angle glaucoma is more common in women. Worldwide, glaucoma is the second-leading cause of blindness after cataracts.

Macular degeneration, also known as age-related macular degeneration (AMD or ARMD), is a medical condition which may result in blurred or no vision in the center of the visual field. Early on there are often no symptoms. Over time, however, some people experience a gradual worsening of vision that may affect one or both eyes. While it does not result in complete blindness, loss of central vision can make it hard to recognize faces, drive, read, or perform other activities of daily life. Visual hallucinations may also occur but these do not represent a mental illness.

Macular degeneration typically occurs in older people. Genetic factors and smoking also play a role. It is due to damage to the macula of the retina. Diagnosis is by a complete eye exam. The severity is divided into early, intermediate, and late types. The late type is additionally divided into “dry” and “wet” forms with the dry form making up 90% of cases.

Preventive efforts include exercising, eating well, and not smoking. Antioxidant vitamins and minerals do not appear to be useful for prevention. There is no cure or treatment that returns vision already lost. In the wet form, anti-VEGF medication injected into the eye or less commonly laser coagulation or photodynamic therapy may slow worsening. Supplements in those who already have the disease may slow progression.

In 2015 it affected 6.2 million people globally. In 2013 it was the fourth most common cause of blindness after cataracts, preterm birth, and glaucoma. It most commonly occurs in people over the age of fifty and in the United States is the most common cause of vision loss in this age group. About 0.4% of people between 50 and 60 have the disease, while it occurs in 0.7% of people 60 to 70, 2.3% of those 70 to 80, and nearly 12% of people over 80 years old.

Retinitis pigmentosa (RP) is a genetic disorder of the eyes that causes loss of vision. Symptoms include trouble seeing at night and decreased peripheral vision (side vision). Onset of symptoms is generally gradual. As peripheral vision worsens, people may experience “tunnel vision.” Complete blindness is uncommon.

Retinitis pigmentosa is generally inherited from a person's parents. Mutations in one of more than 50 genes is involved. The underlying mechanism involves the progressive loss of rod photoreceptor cells in the back of the eye. This is generally followed by loss of cone photoreceptor cells. Diagnosis is by an examination of the retina finding dark pigment deposits. Other supportive testing may include an electroretinogram, visual field testing, or genetic testing.

There is currently no cure for retinitis pigmentosa. Efforts to manage the problem may include the use of low vision aids, portable lighting, or a guide dog. Vitamin A palmitate supplements may be useful to slow worsening. A visual prosthesis may be an option in certain people with severe disease. It is estimated to affect 1 in 4,000 people. Onset is often in childhood but some are not affected until adulthood.

18. Anxiety

Anxiety disorders are a group of mental disorders characterized by significant feelings of anxiety and fear. Anxiety is a worry about future events, and fear is a reaction to current events. These feelings may cause physical symptoms, such as a fast heart rate and shakiness. There are several anxiety disorders, including generalized anxiety disorder, specific phobia, social anxiety disorder, separation anxiety disorder, agoraphobia, panic disorder, and selective mutism. The disorder differs by what results in the symptoms. People often have more than one anxiety disorder.

The cause of anxiety disorders is a combination of genetic and environmental factors. Risk factors include a history of child abuse, family history of mental disorders, and poverty. Anxiety disorders often occur with other mental disorders, particularly major depressive disorder, personality disorder, and substance use disorder. To be diagnosed symptoms typically need to be present for at least 6 months, be more than what would be expected for the situation and decrease functioning. Other problems that may result in similar symptoms include hyperthyroidism; heart disease; caffeine, alcohol, or cannabis use; and withdrawal from certain drugs, among others.

Without treatment, anxiety disorders tend to remain Treatment may include lifestyle changes, counselling, and medications. Counselling is typically with a type of cognitive behavioral therapy. Medications, such as antidepressants, benzodiazepines, or beta blockers, may improve symptoms.

About 12% of people are affected by an anxiety disorder in a given year, and between 5% and 30% are affected over a lifetime. They occur in females about twice as often as in males, and generally begin before age 25 years. The most common are specific phobias, which affect nearly 12%, and social anxiety disorder, which affects 10%. Phobias mainly affect people between the ages of 15 and 35 and become less common after age 55. Rates appear to be higher in the United States and Europe.

19. PTSD

Posttraumatic stress disorder (PTSD) is a mental disorder that can develop after a person is exposed to a traumatic event, such as sexual assault, warfare, traffic collisions, or other threats on a person's life. Symptoms may include disturbing thoughts, feelings, or dreams related to the events, mental or physical distress to trauma-related cues, attempts to avoid trauma-related cues, alterations in how a person thinks and feels, and an increase in the fight-or-flight response. These symptoms last for more than a month after the event. Young children are less likely to show distress, but instead may express their memories through play. A person with PTSD is at a higher risk for suicide and intentional self-harm.

Most people who experience a traumatic event do not develop PTSD. People who experience interpersonal trauma such as rape or child abuse are more likely to develop PTSD, as compared to people who experience non-assault-based trauma, such as accidents and natural disasters. About half of people develop PTSD following rape. Children are less likely than adults to develop PTSD after trauma, especially if they are under 10 years of age. Diagnosis is based on the presence of specific symptoms following a traumatic event.

Prevention may be possible when counselling is targeted at those with early symptoms but is not effective when provided to all trauma-exposed individuals whether or not symptoms are present. The main treatments for people with PTSD are counselling (psychotherapy) and medication. Antidepressants of the selective serotonin reuptake inhibitor type are the first-line medications for PTSD and result in benefit in about half of people. Benefits from medication are less than those seen with counselling. It is not known whether using medications and counselling together has greater benefit than either method separately. Other medications do not have enough evidence to support their use and, in the case of benzodiazepines, may worsen outcomes.

In the United States, about 3.5% of adults have PTSD in a given year, and 9% of people develop it at some point in their life. In much of the rest of the world, rates during a given year are between 0.5% and 1%. Higher rates may occur in regions of armed conflict.^{[2]} It is more common in women than men. Symptoms of trauma-related mental disorders have been documented since at least the time of the ancient Greeks. During the World Wars, the condition was known under various terms including “shell shock” and “combat neurosis.”

20. Other Conditions

Other conditions that may be treated using the cannabis dry powder formulations disclosed herein include for treating neurodermitis, contact eczema, allergies, for the prevention or treatment of phototoxic reactions, for the treatment of conglobata, itching dermatoses, rosacea, perioral dermatitis, acne, acne conglobata, psoriasis (vulgaris, arthropathica, pustulosa), mosquito bites, skin atrophy (in particular also cortisone-related skin changes), allergic rhinitis, privinismus, conjunctivitis, otitis externa, bronchial asthma, tuberculosis sclerosis complex, Lennox-Gastaut syndrome, COPD, Crohn's disease, ulcerative colitis, sarcoidosis, or inflammatory-rheumatic diseases of the soft tissue or joints, graft-versus-host disease, or external mycoses.

### VII. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

A dry powder formulation for inhalation containing 25% (w/w) of Cannabidiol (CBD) was developed using TFF technology, and a pharmacokinetic (PK) study in rats was performed using PennCentury™ DP4 dry powder Insufflator™ with a target delivered dose of 10 mg/kg CBD. The Cₘₐₓ in plasma was 471 ng/mL and Tₘₐₓ was 5 min (the first time point after administration of the inhaled dry powder dose). The CBD level in plasma decreased from 471 to 263 ng/mL between 5 and 15 min, and then remained relatively constant between 116 ng/mL to 246 ng/mL in the time range from 30 minutes to 8 hours. For comparison and in contrast to the plasma levels of this TFF-CBD inhaled powder, in an inhaled CBD pharmacokinetic study in rats reported by Hložek et al. (2017) using CBD dissolved in a ethanolic solution and vaporized using heat, the inventors calculated based on information in this paper the administered amount of CBD as the delivered dose of 1.798 mg, or 7.98 mg/kg (calculated according Wang et al., 2014; DD=C×RMV×D, where DD is the delivered dose in mg, C is the concentration of drug powder in air in mg/L, RMV is the respiratory minute volume of a rat in L/min (RMV (L/min) for rats is calculated as =0.608×BW (kg)^{0.852}), and D is the exposure duration in min; based on 20 mg CBD nebulized into 9.5 L dosing chamber; a calculated RMV of 0.17 L/min, an average body weight of 225 g and exposure time of 5 min). This Hložek et al. (2017) study found that the CBD serum level peaked immediately after removing the rats from their inhalation dosing chamber at about 220 ng/mL followed by a rapid decline to less than about 20 ng/mL baseline by four hours and continued to decline through 24 hours. There is a difference between this TFF-CBD inhaled powder that maintained relatively constant plasma levels over 8 hours compared to the rapid decline in serum levels of inhaled CBD ethanolic solution reported by Hložek et al. (2017). Possible explanations include that the TFF-CBD powder can better avoid lung clearance and that the TFF-CBD powder particles deposited in the lungs continue to dissolve and be absorbed from the lungs to plasma. (Beinborn et al., 2012; Wang et al., 2014; Carvalho et al., 2014). Also, CBD amount in rat lungs following dosing with TFF-CBD after 8 hours in the inventors' PK study ranged from 412 μg to 1.78 mg (average 831.4±508.1 μg), indicating that TFF-CBD is still available in the lungs for absorption systemically. Based on these results, the target delivered dose (10 mg/kg) of TFF-CBD in this study can be reduced or increased depending on the therapeutic indication desired. For comparison, based on CBD plasma levels of Huntington's disease patients, as reported by Consroe et al. (1991), the orally administered CBD dose of 10 mg/kg/day, which is the recommended maintenance dose of Epidiolex®, for 6 weeks by oral administration resulted a steady state CBD plasma level between 5.9 and 11.2 ng/mL. Notably, Consroe et al. (1991) reports that for Huntington's disease their dosing level was neither symptomatically effective nor toxic. The present study produced TFF-CBD plasma level in rats of about 200 ng/mL, and in this study produces CBD inhaled powder that achieves higher and more sustained plasma levels following inhalation of the dry powder at the same mg/kg dose as compared to IV administration of a CBD solution.

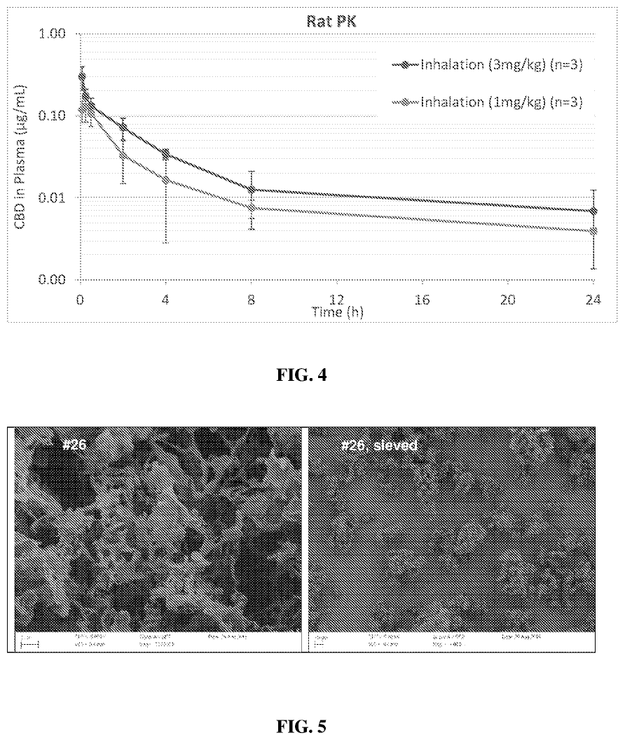

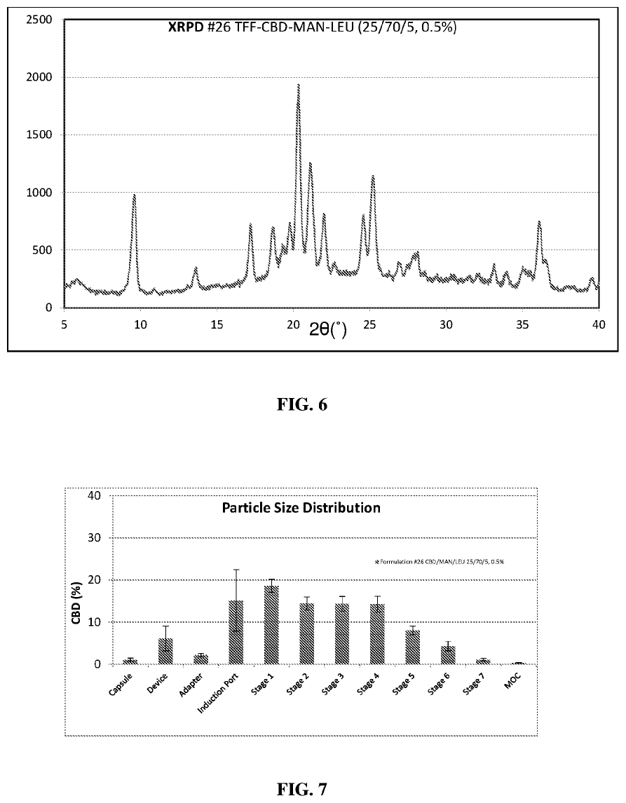

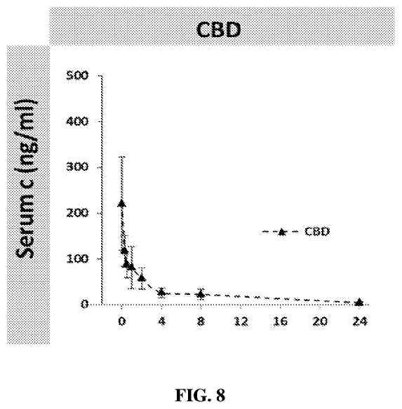

To study of plasma level with lower dose of the TFF-CBD powder by dry powder inhalation, another pharmacokinetic study in rats was performed using PennCentury™ DP4 dry powder Insufflator™ with a target delivered dose of 3 mg/kg and 1 mg/kg CBD. While the Cₘₐₓ in plasma with 3 mg/kg dose was 301 ng/mL and Tₘₐₓ was 5 min (the first time point after administration of the inhaled dry powder dose), the Cₘₐₓ in plasma with 1 mg/kg dose was 118 ng/mL and Tₘₐₓ was 15 min (the second time point after administration of the inhaled dry powder dose) (see FIG. 4). When compared to vaporized CBD liquid as CBD serum level in rat pharmacokinetic study reported by Hložek et al. (2017), the CBD levels in rat plasma with the TFF-CBD dry powder inhalation of 1 mg/kg dose was comparable with the CBD levels in rat serum to that of vaporized CBD delivered at a much higher dose of about 8 mg/kg. In other words, TFF-CBD dry powder inhalation was more efficient in delivering CBD than the vaporized CBD liquid. Therefore, delivery of TFF-CBD powder by dry powder inhalation requires a lower dose to reach the similar systemic levels of CBD as compared to vaporized CBD liquid by inhalation, thus showing TFF-CBD will present better efficacy at equivalent doses.

The inventors also note that in this study that the inhaled TFF-CBD was well tolerated in rats, and the rats did not show any agitation or abnormal activity after administration of inhaled TFF-CBD during the test period of time.

TABLE 1
List of TFF-Cannabinoids formulations made by TFF*

| | | Solid | | Processing |
|---|---|---|---|---|
| Formulation | | Loading | | Temperature |
| Number | Compositions (w/w) | (% w/v) | Solvent (v/v) | (° C.) |
| 1 | CBD | 1 | Water/ACN (40/60) | −150 |
| 2 | CBD/MAN (75/25) | 1 | Water/ACN (40/60) | −150 |
| 3 | CBD/LAC (75/25) | 1 | Water/ACN (40/60) | −150 |
| 4 | CBD/LEU (75/25) | 1 | Water/ACN (40/60) | −150 |
| 5 | CBD/LEC (75/25) | 1 | Water/ACN (40/60) | −150 |
| 6 | CBD/MAN (50/50) | 0.5 | Water/ACN (40/60) | −130 |
| 7 | CBD/MAN (25/75) | 0.5 | Water/ACN (40/60) | −130 |
| 8 | CBD/LAC (50/50) | 0.5 | Water/ACN (40/60) | −130 |
| 9 | CBD/LAC (25/75) | 0.5 | Water/ACN (40/60) | −130 |
| 10 | CBD/LEU (50/50) | 0.5 | Water/ACN (40/60) | −130 |
| 11 | CBD/LEU (25/75) | 0.5 | Water/ACN (40/60) | −130 |
| 12 | CBD/PVP K12 (25/75) | 0.5 | 1,4-dioxane | −130 |
| 13 | CBD/PVP K25 (25/75) | 0.5 | 1,4-dioxane | −130 |
| 14 | CBD/PVP K30 (25/75) | 0.5 | 1,4-dioxane | −130 |
| 15 | CBD/copovidone (25/75) | 0.5 | 1,4-dioxane | −130 |
| 16 | CBD/HPMC (25/75) | 0.5 | 1,4-dioxane | −130 |
| 17 | CBD/HPMC-AS (25/75) | 0.5 | 1,4-dioxane | −130 |
| 18 | CBD/LAC (25/75) | 0.75 | Water/ACN (40/60) | −130 |
| 19 | CBD/LAC/LEU (25/70/5) | 0.5 | Water/ACN (40/60) | −130 |
| 20 | CBD/LAC/LEU (25/70/5) | 0.75 | Water/ACN (40/60) | −130 |
| 21 | CBD/LAC (40/60) | 0.5 | Water/ACN (40/60) | −130 |
| 22 | CBD/LAC (40/60) | 0.75 | Water/ACN (40/60) | −130 |
| 23 | CBD/LAC/LEU (40/55/5) | 0.5 | Water/ACN (40/60) | −130 |
| 24 | CBD/LAC/LEU (40/55/5) | 0.75 | Water/ACN (40/60) | −130 |
| 25 | CBD/LAC/LEU (50/45/5) | 0.5 | Water/ACN (40/60) | −130 |
| 26 | CBD/MAN/LEU (25/70/5) | 0.5 | Water/ACN (40/60) | −130 |
| 27 | CBD/TRE (25/75) | 0.5 | Water/ACN (40/60) | −130 |

Abbreviations:
Cannabidiol (CBD),
mannitol (MAN),
lactose (LAC),
L-leucine (LEU),
lecithin (LEC),
trehalose (TRE)
*Formulations 1-27 were prepared using thin-film freezing technology. The exemplary cannabinoid, CBD, and excipient(s) are dissolved in solvent in the amounts given in Table 1. The solution was applied onto a rotating stainless-steel drum, which was cryogenically cooled at the listed temperature in Table 1. The frozen samples were collected in a stainless-steel tray containing liquid nitrogen. The samples were then stored at −80° C. freezer to remove excess liquid nitrogen, then solvent was removed by sublimation to produce a powder for dry powder inhalation.

TABLE 2
Aerodynamic properties of TFF-Cannabinoids*

| | | | | FPF | FPF | Delivered |
|---|---|---|---|---|---|---|
| Formulation | | MMAD | GSD | (% of | (% of | dose |
| number | Compositions | (μm) | (μm) | recovered) | delivered) | (%) |
| 7 | CBD/MAN 25/75 | 3.88 | 2.19 | 30.37 | 40.95 | 75.76 |
| 9 | CBD/LAC 25/75 | 2.52 | 1.93 | 60.54 | 69.17 | 91.47 |
| 11 | CBD/LEU 25/75 | 0.73 | 2.98 | 83.91 | 94.09 | 91.87 |
| 13 | CBD/PVP K25 25/75 | 2.05 | 2.36 | 70.37 | 80.15 | 92.61 |
| 26 | CBD/MAN/LEU | 3.59 | 2.59 | 47.29 | 51.14 | 92.77 |
| | 25/70/5 | | | | | |

*Aerosol properties of TFF-CBD powders for inhalation in exemplary formulations 7, 9, 11, 13 and 26, prepared as described in Example 1. FPF is the amount of drug recovered that is less than 5 microns in aerodynamic diameter and is reported as either % recovered dose or as % delivered dose. The % recovered dose (i.e., % metered dose) refers to the drug recovered from the capsule, device, adapter, induction port and all states of the impactor. The % delivered dose (i.e., % emitted dose) refers to the drug recovered that is emitted and does not include drug retained in the capsule and device (so, it only includes drug from the adapter, induction port and all stages of the impactor).

TABLE 3
Cannabidiol concentrations in rat plasma*

| | | Inhalation | Intravenous | Inhalation |
|---|---|---|---|---|
| | Intravenous | (10 mg/kg) | (3 mg/kg) | (1 mg/kg) |
| | (ng/mL) | (ng/mL) | (ng/mL) | (ng/mL) |
| Time(h) | (n = 5) | (n = 6) | (n = 3) | (n = 3) |
| 0.083 | 4279.4 ± 891.0 | 470.8 ± 337.8 | 300.8 ± 93.3 | 117.5 ± 33.3 |
| 0.25 | 1766.2 ± 296.8 | 263.4 ± 105.2 | 173.3 ± 38.1 | 129.2 ± 45.6 |
| 0.5 | 1041.9 ± 217.3 | 194.7 ± 76.4 | 131.7 ± 31.3 | 108.5 ± 34.7 |
| 1 | 600.9 ± 192.8 | 191.4 ± 112.0 | n/a | n/a |
| 2 | 271.5 ± 112.3 | 214.2 ± 106.4 | 71.5 ± 21.4 | 32.9 ± 18.0 |
| 4 | 50.0 ± 23.0 | 111.5 ± 42.6 | 34.1 ± 4.9 | 16.5 ± 13.7 |
| 8 | 16.1 ± 11.3 | 245.9 ± 94.0 | 12.6 ± 8.4 | 7.5 ± 1.9 |
| 24 | n/a | n/a | 6.9 ± 5.5 | 3.9 ± 0.3 |

*A pharmacokinetic study was performed in rats on exemplary formulation # 26 dry powder suitable for inhalation. For comparison, an intravenous solution containing 8 mg/mL CBD in 5% (v/v) ethanol, 4% (v/v) Cremophor EL, 6% (v/v) Tween 80, and 0.1% (w/v) ascorbic acid in saline solution was tested for comparison. The intravenous injection was given via a catheter. The study was performed in 6 rats by dry powder inhalation administered by a PennCentury ™ DP4 insufflator ™ (10 mg/kg), and 5 rats for intravenous administration (10 mg/kg). The dry powder formulation was sieved using a sieve no. 200, size 75 μm before loading to DP4 device. 250 μL blood sample was taken from the rats at each time point, and plasma was separated by centrifugation. Acetonitrile (600 μL) was added to 100 μL of plasma sample and vortexed for 30 seconds. Water (3.0 mL) was added to the sample and vortexed for 30 seconds. Ethyl acetate (3.0 mL) was added to the sample and vortexed for 1 minute. The sample was then centrifuged for 15 minutes at 4000 rpm at 10° C. The organic layer was separated, dried, and reconstituted with 200 μL of diluent (water/acetonitrile 30/70 v/v) to analyze the quantity of CBD by HPLC/UV at 210 nm. The pharmacokinetic data are given in Table 3.

### Example 2

CBD dry powder formulation for inhalation containing terpenes, which are aromatic compounds that evaporate easily and readily are sensed by the nose, and are found in fruits, flowers, and all herbs including hemp, was developed. Terpenes are being included as an example of synergistic therapeutic effect with the cannabinoids, reinforcing experience by affecting the sensory experience of the CBD user, including taste, flavor and the like. Thus, terpenes can provide an entourage effect, or in other words, a synergistic benefit when terpenes are added to cannabinoids, that enforces therapeutic benefits like relaxation. Terpene stock solution was prepared by dispersing terpene in ethanol, acetonitrile, and ethyl acetate at concentrations of 20 mg/mL. The terpene stock solution was then added to Lactohale® 300 (LH300) at terpene amount of 2.5 or 5.0% (w/w). The solvents in the mixture was removed in an oven at 40° C. for 15 to 45 minutes. LH300 containing terpene was blended with CBD dry powder formulation for inhalation (formulation 26) by either rolling or V shape blender to produce CBD dry powder formulation for inhalation containing terpene. While the highest recovery of terpene in LH300 was obtained by using solvent of acetonitrile or ethyl acetate at a concentration of 2.5% (w/w) that yielded over 50%, the highest amount of terpene in LH300 was made with 5.0% (w/w) terpene in either ethanol or ethyl acetate with over 1.8% (w/w) terpene in LH300. The amount of terpene in LH300 was determined by HPLC/UV at a detection wavelength of 205 nm.

TABLE 4
Terpene concentration with LH300

| | Theoretical | Experimental | Recovered Amount of |
|---|---|---|---|
| | Amount of | Amount of | Terpene |
| Solvent for | Terpene with | Terpene with | (experimental/ |
| Stock Terpene | LH300 | LH300 | theoretical) |
| Solution | (% w/w) | (% w/w) | (%) |
| Ethanol | 2.5 | 0.84 ± 0.02 | 33.3 |
| | 5.0 | 1.86 ± 0.0.3 | 37.0 |
| Acetonitrile | 2.5 | 1.28 ± 0.09 | 51.5 |
| | 5.0 | 1.31 ± 0.01 | 26.4 |
| Ethyl acetate | 2.5 | 1.34 ± 0.02 | 52.7 |
| | 5.0 | 1.83 ± 0.02 | 35.8 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. References

The following references, to the extent that they provide exemplary procedural or other details supplementary to those set forth herein, are specifically incorporated herein by reference.
Beinborn N A, Du J, Wiederhold N P, Smyth H D, Williams R O, 3rd. Dry powder insufflation of crystalline and amorphous voriconazole formulations produced by thin film freezing to mice. Eur J Pharm Biopharm. 2012; 81(3):600-8.
Carvalho S R, Watts A B, Peters J I, Liu S, Hengsawas S, Escotet-Espinoza M S, et al. Characterization and pharmacokinetic analysis of crystalline versus amorphous rapamycin dry powder via pulmonary administration in rats. Eur J Pharm Biopharm. 2014; 88(1):136-47.
Consroe P, Laguna J, Allender J, Snider S, Stern L, Sandyk R, et al. Controlled clinical trial of cannabidiol in Huntington's disease. Pharmacol Biochem Behav. 1991; 40(3):701-8.
Hlozek T, Uttl L, Kaderabek L, Balikova M, Lhotkova E, Horsley R R, et al. Pharmacokinetic and behavioural profile of THC, CBD, and THC+CBD combination after pulmonary, oral, and subcutaneous administration in rats and confirmation of conversion in vivo of CBD to THC. Eur Neuropsychopharmacol. 2017; 27(12):1223-37.
Wang Y-B, Watts A B, Peters J I, Liu S, Batra A, Williams R O, 3rd. In vitro and in vivo performance of dry powder inhalation formulations: comparison of particles prepared by thin film freezing and micronization. AAPS PharmSciTech. 2014; 15(4):981-93.

1. A thin film freezing (TFF) preparation comprising one or more cannabinoids, one or more excipients, and optionally further comprising one or more inactive processing agents.

2. The TFF preparation of claim 1, wherein the preparation is dry powder composition.

3. The TFF preparation of claim 1, wherein the preparation is a pharmaceutical inhalation composition.

4. The TFF preparation of claim 1, wherein the one or more cannabinoids in the preparation is/are amorphous.

5. The TFF preparation of claim 1, wherein the preparation is amorphous.

6. The TFF preparation of claim 1, wherein the one or more cannabinoids in the preparation is/are crystalline.

7. The TFF preparation of claim 1, wherein the preparation is a crystalline preparation.

8. The TFF preparation of claim 1, wherein the preparation is a mixture of crystalline and amorphous preparations.

9. The TFF preparation of claim 1, wherein the one or more excipients comprises a sugar or sugar derivative, such as mannitol, trehalose, lactose, sucrose, maltose, a starch, cellulose, xylitol, sorbitol, erythritol, threitol, arabitol, ribitol, galactitol, fucitol, iditol, inositol, volemitol, isomalt, maltitol, lactitol, maltotritol, maltotetraitol, polyglycitol, or maltodextrin.

10. The TFF preparation of claim 1, wherein the one or more inactive processing agents are an amino acid or an amino acid derivative such as leucine, arginine, glycine, isoleucine, lysine, valine, methionine, phenylalanine, aspartame, acesulfame K; or non-amino acid or amino acid derivatives such as zinc stearate, magnesium stearate, calcium stearate, povidone K25, or sodium stearate.

11. The TFF preparation of claim 1, further comprising a lung surfactant, such as wherein the lung surfactant comprises one or more of lecithin, oleic acid, lauric acid, palmitic acid, stearic acid, erucic acid, behenic acid, dipalmitoyl phosphatidylcholine (DPPC), dipalmitoyl phosphatidylethanolamine (DPPE), dipalmitoyl phosphatidylinositol (DPPI), phosphatidylcholines, phosphatidylethanolamines, phosphatidylglycerols, sodium lauryl sulphate, magnesium lauryl sulphate, or cholesterol.

12. The TFF preparation of claim 1, wherein the ratio of cannabinoids:excipient:inactive processing agent is at a ratio of 10-90:10-90:0-90, a ratio of 25-35:65-75:0:10, or ratio of 25:70:5.

13. The TFF preparation of claim 1, wherein the mass median aerodynamic diameter (MMAD) of the particles is from about 0.5 to about 8 microns, more preferably from about 1 to about 5 microns.

14. The TFF preparation of claim 1, wherein one or more cannabinoids is/are plant-based cannabinoid or synthetic cannabinoid or mixtures thereof.

15. The TFF preparation of claim 1, wherein the preparation further comprises tetrahydro-cannabinol.

16. The TFF preparation of claim 1, wherein the one or more cannabinoids comprises cannabidiol (CBD), for example, where the preparation comprises CBD:mannitol:leucine at a ratio of 25:70:5.

17. The TFF preparation of claim 1, further comprising a terpene.

18. A method comprising administering a TFF preparation according to claim 1 via inhalation.

19. The method of claim 18, wherein the subject has a pulmonary disease, such as COPD or asthma.

20. The method of claim 18, wherein the subject has a neurological disease or disorder, such as Alzheimer's disease, epilepsy, an autism spectrum disorder, PTSD, Parkinson's disease, Huntington's disease, schizophrenia, stroke, major depression or traumatic brain injury.

21. The method of claim 18, wherein the subject has ocular disease, such as macular degeneration, glaucoma or retinitis pigmentosa (RP).

22. The method of claim 18, wherein the subject has cancer, Rett syndrome (RTT), Lennox-Gastaut Syndrome (LGS), Tuberous Sclerosis Complex (TSC), Dravet syndrome, nausea, anxiety, pain, dystonia, diabetes, Rheumatoid arthritis, Crohn's disease, graft-versus-host disease (GVHD) or HIV infection.

23. The method of claim 16, wherein administering uses a dry powder inhaler device comprising said TFF preparation.

24. The method of claim 16, wherein the TFF preparation is administered 1, 2, 3, 4, 5, 6, 7, 8, 9, 10, 11, 12, 13 or 14 times a week or 1, 2, 3, 4, 5 or 6 times per day, or is administered on a chronic basis.

25. The method of claim 16, wherein said subject is treated with at least another therapy.

26. The method of claim 25, wherein the other therapy is (a) given through a pulmonary route, (b) given through a non-pulmonary route, (c) given before, at the same time or after the TFF preparation, (d) co-formulated with the TFF preparation, or (e) not co-formulated with the TFF preparation.

27. The method of claim 25, wherein the other therapy is a second inhalation therapy, and the second inhalation therapy is administered using the same dry powder inhaler as the TFF preparation but in a distinct compartment from said TFF preparation.

28. The method of claim 16, wherein a single dose of said TFF preparation is 0.1 mg to 100 mg.

29. The method of claim 16, wherein the total dose of said TFF preparation is 0.1 to 10 g.

30. A dry powder inhaler comprising a TFF preparation according to claim 1.

31. A method of preparing a thin film freezing (TFF) preparation comprising one or more cannabinoids, one or more excipients, and optionally one or more inactive processing agents comprising:
(a) mixing one or more cannabinoids, one or more excipients, and one or more optional inactive processing agents in one or more solvents to produce a solution;
(b) applying said solution to a rotating drum cooled to −60° C. or lower to produce a frozen solution;
(c) collecting the frozen solution in liquid nitrogen, mechanical cooling or in a tray cooled by dry ice;
(d) storing said frozen solution at −80° C. to remove residue liquid nitrogen; and
(e) removing solvent by sublimation.

32. The method of claim 31, wherein the preparation further comprises a lung surfactant.

33. The method of claim 31, wherein the preparation further comprises a terpene.

34. The method of claim 31, wherein the one or more cannabinoids comprises cannabidiol (CBD), for example, where the preparation comprises CBD:mannitol:leucine at a ratio of 25:70:5.
